SEQUENCE LISTING (9 sequences)

SEQ ID NO: 1 - RNA, 1065 nt, Artificial Sequence
  ORF human  OTC mRNA codon optimized for human expression
augcuguuuaaccugaggauucugcugaacaacgcugcuuuucggaacggccacaacuuuauggugcggaacuuucggugcggacagccacugcagaacaaagugcagcugaaggggagggaccugcugacccugaaaaauuucacaggagaggaaaucaaguacaugcuguggcugucugccgaucugaaguuccggaucaagcagaagggcgaauaucugccacugcugcagggcaaaagucuggggaugaucuucgaaaagaggaguacucggaccagacugucaacagagacuggauucgcucugcugggaggacacccaugcuuucugaccacacaggacauucaucugggcgugaacgagucacugaccgacacagcucgaguccugagcuccauggcagaugccgugcuggcacgggucuacaaacagagcgaccuggauacccuggcuaaggaagcaagcauccccaucauuaaugggcuguccgaccuguaucacccuauccagauucuggccgauuaccugacccugcaggagcauuauucuagucugaaaggccugacacugagcuggauuggggacggaaacaauauccugcacuccauuaugaugucugccgcuaaguuuggaaugcaucugcaggcagccacaccaaaaggcuacgaacccgaugccagugugacuaagcuggccgaacaguaugcuaaagagaacggcacuaagcugcugcugaccaaugacccucuggaggcugcacacggaggcaacguccugaucacugauaccuggauuuccaugggccaggaggaagagaagaaaaagcgccugcaggcauuccagggguaccaggugacaaugaaaacugccaaggucgccgcuucugauuggacuuuucugcauugucugccccgaaaaccugaagagguggacgaugaggucuucuauucaccuaggagccugguguuuccagaagccgagaaucgcaaguggacaaucauggcugugauggugucccugcugacugauuauuccccccagcugcagaaaccuaaguucuga

SEQ ID NO: 2 - RNA, 47 nt, Artificial Sequence
  5′ UTR
aggaaauaagagagaaaagaagaguaagaagaaauauaagagccacc

SEQ ID NO: 3 - RNA, 114 nt, Artificial Sequence
  3′ UTR
gcggccgcuuaauuaagcugccuucugcggggcuugccuucuggccaugcccuucuucucucccuugcaccuguaccucuuggucuuugaauaaagccugaguaggaagucuag

SEQ ID NO: 4 - RNA, 1226 nt, Artificial Sequence
  5′ UTR, ORF human  OTC mRNA codon optimized for human expression, 3′ UTR
aggaaauaagagagaaaagaagaguaagaagaaauauaagagccaccaugcuguuuaaccugaggauucugcugaacaacgcugcuuuucggaacggccacaacuuuauggugcggaacuuucggugcggacagccacugcagaacaaagugcagcugaaggggagggaccugcugacccugaaaaauuucacaggagaggaaaucaaguacaugcuguggcugucugccgaucugaaguuccggaucaagcagaagggcgaauaucugccacugcugcagggcaaaagucuggggaugaucuucgaaaagaggaguacucggaccagacugucaacagagacuggauucgcucugcugggaggacacccaugcuuucugaccacacaggacauucaucugggcgugaacgagucacugaccgacacagcucgaguccugagcuccauggcagaugccgugcuggcacgggucuacaaacagagcgaccuggauacccuggcuaaggaagcaagcauccccaucauuaaugggcuguccgaccuguaucacccuauccagauucuggccgauuaccugacccugcaggagcauuauucuagucugaaaggccugacacugagcuggauuggggacggaaacaauauccugcacuccauuaugaugucugccgcuaaguuuggaaugcaucugcaggcagccacaccaaaaggcuacgaacccgaugccagugugacuaagcuggccgaacaguaugcuaaagagaacggcacuaagcugcugcugaccaaugacccucuggaggcugcacacggaggcaacguccugaucacugauaccuggauuuccaugggccaggaggaagagaagaaaaagcgccugcaggcauuccagggguaccaggugacaaugaaaacugccaaggucgccgcuucugauuggacuuuucugcauugucugccccgaaaaccugaagagguggacgaugaggucuucuauucaccuaggagccugguguuuccagaagccgagaaucgcaaguggacaaucauggcugugauggugucccugcugacugauuauuccccccagcugcagaaaccuaaguucugagcggccgcuuaauuaagcugccuucugcggggcuugccuucuggccaugcccuucuucucucccuugcaccuguaccucuuggucuuugaauaaagccugaguaggaagucuag

SEQ ID NO: 5 - RNA, 1311 nt, Artificial Sequence
  5′ UTR, ORF human  OTC mRNA codon optimized for human expression, 3′ UTR, and polyA (80)
aggaaauaagagagaaaagaagaguaagaagaaauauaagagccaccaugcuguuuaaccugaggauucugcugaacaacgcugcuuuucggaacggccacaacuuuauggugcggaacuuucggugcggacagccacugcagaacaaagugcagcugaaggggagggaccugcugacccugaaaaauuucacaggagaggaaaucaaguacaugcuguggcugucugccgaucugaaguuccggaucaagcagaagggcgaauaucugccacugcugcagggcaaaagucuggggaugaucuucgaaaagaggaguacucggaccagacugucaacagagacuggauucgcucugcugggaggacacccaugcuuucugaccacacaggacauucaucugggcgugaacgagucacugaccgacacagcucgaguccugagcuccauggcagaugccgugcuggcacgggucuacaaacagagcgaccuggauacccuggcuaaggaagcaagcauccccaucauuaaugggcuguccgaccuguaucacccuauccagauucuggccgauuaccugacccugcaggagcauuauucuagucugaaaggccugacacugagcuggauuggggacggaaacaauauccugcacuccauuaugaugucugccgcuaaguuuggaaugcaucugcaggcagccacaccaaaaggcuacgaacccgaugccagugugacuaagcuggccgaacaguaugcuaaagagaacggcacuaagcugcugcugaccaaugacccucuggaggcugcacacggaggcaacguccugaucacugauaccuggauuuccaugggccaggaggaagagaagaaaaagcgccugcaggcauuccagggguaccaggugacaaugaaaacugccaaggucgccgcuucugauuggacuuuucugcauugucugccccgaaaaccugaagagguggacgaugaggucuucuauucaccuaggagccugguguuuccagaagccgagaaucgcaaguggacaaucauggcugugauggugucccugcugacugauuauuccccccagcugcagaaaccuaaguucugagcggccgcuuaauuaagcugccuucugcggggcuugccuucuggccaugcccuucuucucucccuugcaccuguaccucuuggucuuugaauaaagccugaguaggaagucuagaaaaaaaaaaaaaaaaaaaaaaaaaaaaaaaaaaaaaaaaaaaaaaaaaaaaaaaaaaaaaaaaaaaaaaaaaaaaaaaaaagcu

SEQ ID NO: 6 - RNA, 1666 nt, Artificial Sequence
  5′ UTR, ORF human  OTC mRNA codon optimized for human expression, 3′ UTR, and polyA
aggaaauaagagagaaaagaagaguaagaagaaauauaagagccaccaugcuguuuaaccugaggauucugcugaacaacgcugcuuuucggaacggccacaacuuuauggugcggaacuuucggugcggacagccacugcagaacaaagugcagcugaaggggagggaccugcugacccugaaaaauuucacaggagaggaaaucaaguacaugcuguggcugucugccgaucugaaguuccggaucaagcagaagggcgaauaucugccacugcugcagggcaaaagucuggggaugaucuucgaaaagaggaguacucggaccagacugucaacagagacuggauucgcucugcugggaggacacccaugcuuucugaccacacaggacauucaucugggcgugaacgagucacugaccgacacagcucgaguccugagcuccauggcagaugccgugcuggcacgggucuacaaacagagcgaccuggauacccuggcuaaggaagcaagcauccccaucauuaaugggcuguccgaccuguaucacccuauccagauucuggccgauuaccugacccugcaggagcauuauucuagucugaaaggccugacacugagcuggauuggggacggaaacaauauccugcacuccauuaugaugucugccgcuaaguuuggaaugcaucugcaggcagccacaccaaaaggcuacgaacccgaugccagugugacuaagcuggccgaacaguaugcuaaagagaacggcacuaagcugcugcugaccaaugacccucuggaggcugcacacggaggcaacguccugaucacugauaccuggauuuccaugggccaggaggaagagaagaaaaagcgccugcaggcauuccagggguaccaggugacaaugaaaacugccaaggucgccgcuucugauuggacuuuucugcauugucugccccgaaaaccugaagagguggacgaugaggucuucuauucaccuaggagccugguguuuccagaagccgagaaucgcaaguggacaaucauggcugugauggugucccugcugacugauuauuccccccagcugcagaaaccuaaguucugagcggccgcuuaauuaagcugccuucugcggggcuugccuucuggccaugcccuucuucucucccuugcaccuguaccucuuggucuuugaauaaagccugaguaggaagucuagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 7 - protein, 354 aa, Artificial Sequence
  human  OTC
MLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGFALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 8 - DNA, 1226 bp, Artificial Sequence
  DNA sequence for 3′ UTR, human OTC open reading frame and 5′ UTR
aggaaataagagagaaaagaagagtaagaagaaatataagagccaccatgctgtttaacctgaggattctgctgaacaacgctgcttttcggaacggccacaactttatggtgcggaactttcggtgcggacagccactgcagaacaaagtgcagctgaaggggagggacctgctgaccctgaaaaatttcacaggagaggaaatcaagtacatgctgtggctgtctgccgatctgaagttccggatcaagcagaagggcgaatatctgccactgctgcagggcaaaagtctggggatgatcttcgaaaagaggagtactcggaccagactgtcaacagagactggattcgctctgctgggaggacacccatgctttctgaccacacaggacattcatctgggcgtgaacgagtcactgaccgacacagctcgagtcctgagctccatggcagatgccgtgctggcacgggtctacaaacagagcgacctggataccctggctaaggaagcaagcatccccatcattaatgggctgtccgacctgtatcaccctatccagattctggccgattacctgaccctgcaggagcattattctagtctgaaaggcctgacactgagctggattggggacggaaacaatatcctgcactccattatgatgtctgccgctaagtttggaatgcatctgcaggcagccacaccaaaaggctacgaacccgatgccagtgtgactaagctggccgaacagtatgctaaagagaacggcactaagctgctgctgaccaatgaccctctggaggctgcacacggaggcaacgtcctgatcactgatacctggatttccatgggccaggaggaagagaagaaaaagcgcctgcaggcattccaggggtaccaggtgacaatgaaaactgccaaggtcgccgcttctgattggacttttctgcattgtctgccccgaaaacctgaagaggtggacgatgaggtcttctattcacctaggagcctggtgtttccagaagccgagaatcgcaagtggacaatcatggctgtgatggtgtccctgctgactgattattccccccagctgcagaaacctaagttctgagcggccgcttaattaagctgccttctgcggggcttgccttctggccatgcccttcttctctcccttgcacctgtacctcttggtctttgaataaagcctgagtaggaagtctag

SEQ ID NO: 9 - DNA, 17 bp, Artificial Sequence
  T7 Promoter
taatacgactcactata

(19) United States
(12) Patent Application Publication
(10) Pub. No.: US 2023/0001021 A1
(43) Pub. Date: Jan. 5, 2023

(54) ORNITHINE TRANSCARBAMYLASE (OTC) CONSTRUCTS AND METHODS OF USING THE SAME

(71) Applicants: Mary Prieve, Cambridge, MA (US); Genevant Sciences GmbH, Basel (CH)

(72) Inventors: Owen DALY, Cambridge, MA (US); Kieu LAM, Cambridge, MA (US); James HEYES, Cambridge, MA (US); Richard HOLLAND, Cambridge, MA (US); Christine ESAU, Cambridge, MA (US); Ed YAWORSKI, Cambridge, MA (US); Mary PRIEVE, Cambridge, MA (US)

(21) Appl. No.: 17/771,421

(22) PCT Filed: Oct. 22, 2020
(86) PCT No.: PCT/US2020/056890

§ 371 Date: Apr. 22, 2022

Related U.S. Application Data
(60) Provisional application No. 62/924,567, filed on Oct. 22, 2019.

(51) Int. Cl. A61K 48/00; A61P 1/16; C12N 9/10; A61K 47/69; C12N 15/85

(52) U.S. Cl. CPC A61K 48/0066; A61K 48/0025; A61P 1/16; C12N 9/1018; A61K 47/6911; C12N 15/8509; C12Y 201/03003

(57) ABSTRACT

The present disclosure provides, among other things, polynucleotide constructs, compositions, and methods of treating ornithine transcarbamylase deficiency, including administering to a subject in need thereof a composition comprising a polynucleotide construct comprising a 5′ UTR, a codon optimized mRNA encoding an ornithine transcarbamylase, and a 3′ UTR.

### CROSS REFERENCE TO RELATED APPLICATION

This application claims priority to U.S. Provisional Appl. No. 62/924,567 filed Oct. 22, 2019, the content of which is hereby incorporated by reference in its entirety.

### SEQUENCE LISTING

The content of the electronically submitted sequence listing in ASCII text file (Name: 4170_021PC01_Seqlisting_ST25; Size: 13,467 bytes; and Date of Creation: Oct. 21, 2020) filed with the application is incorporated herein by reference in its entirety.

### BACKGROUND

Ornithine transcarbamylase deficiency (OTC deficiency or OTCD) is an X-linked genetic disorder characterized by complete or partial lack of functional ornithine transcarbamylase (OTC) enzyme, which is typically the result of mutations in the OTC gene. Mutations in the OTC gene can eliminate or reduce the ability of the OTC enzyme to catalyze the synthesis of citrulline (Cit) and phosphate (Pᵢ) (in the liver and small intestine) from carbamoyl phosphate (CP) and ornithine (Orn). This dysfunction in the Urea Cycle can lead to excess ammonia, which can accumulate in the blood (hyperammonemia) and travel to the nervous system, resulting in symptoms associated with OTC deficiency.

OTC deficiency is the most common type of urea cycle disorder. Hundreds of mutations in human OTC have been reported. The severity and age of onset of OTC deficiency vary from person to person, even with in the same family and/or with the same causative mutation. A severe form of the disorder affects some infants, typically males, shortly after birth. A milder form of the disorder affects some children later in infancy. Both males and females can develop symptoms of OTC deficiency during childhood.

Currently, there is no cure other than liver transplant for people with OTC deficiency and long-term therapy involves life-long restriction of protein intake and nitrogen scavenger therapy (e.g., sodium phenyl acetate or sodium phenyl butyrate and/or sodium benzoate). Liver transplantation can also be considered in patients with severe, neonatal-onset OTC deficiency or those with frequent hyperammonemic episodes

RNA molecules have the capacity to act as potent modulators of gene expression in vitro and in vivo and therefore have potential as nucleic acid based drugs. These molecules can function through a number of mechanisms utilizing either specific interactions with cellular proteins or base pairing interactions with other RNA molecules. For disorders characterized by insufficient or faulty protein production, therapeutic mRNA has the potential to provide instructions for ribosomes to produce the missing or faulty protein. Efficient and effective intracellular delivery of RNA therapeutics is difficult because these therapeutics are prone to rapid degradation and excretion in the bloodstream and do not pass freely through cell membranes.

The delivery of exogenous polynucleotides such as RNA molecules and other membrane impermeable compounds into living cells is highly restricted by the complex membrane systems of the cell. Typically, molecules used in antisense and gene therapies are large, negatively charged and hydrophilic molecules. These characteristics can preclude their direct diffusion across the cell membrane to the cytoplasm. Thus, a major barrier to the therapeutic use of polynucleotides for modulation of gene expression is the delivery of the polynucleotide to the cytoplasm. Transfection agents typically comprise peptides, polymers, and lipids of a cationic nature as well as nano- and microparticles. These transfection agents have been used successfully in in vitro reactions. However, there are challenges with efficacy and toxicity in vivo. Furthermore, the cationic charge of these systems can cause interaction with serum components, which causes destabilization of polynucleotide-transfection reagent interaction and poor bioavailability and targeting. When transfecting nucleic acids in vivo, the delivery agent should protect the nucleic acid payload from early extracellular degradation, e.g., from nucleases. Furthermore, the delivery agent should not be recognized by the adaptive immune system (immunogenicity) and should not stimulate an acute immune response.

### BRIEF SUMMARY

The present disclosure provides polynucleotide constructs comprising, from 5′ to 3′: a 5′ UTR comprising the sequence of SEQ ID NO: 2; an mRNA sequence comprising an open reading frame (ORF) encoding a functional human ornithine transcarbamylase (OTC), wherein ORF comprises a codon optimized sequence at least about 95% identical to SEQ ID NO: 1; and a 3′ UTR comprising the sequence of SEQ ID NO: 3.

In certain aspects the disclosure provides polynucleotide constructs comprising an mRNA sequence comprising an open reading frame (ORF) encoding a functional human ornithine transcarbamylase (OTC), wherein the mRNA sequence comprises a sequence having no more than five nucleic acids different from SEQ ID NO: 4. In some aspects, the polynucleotide construct comprises, from 5′ to 3′: a 5′ UTR; the mRNA sequence comprising the ORF encoding the OTC; and a 3′ UTR. In certain aspects, the 5′ UTR comprises the sequence of SEQ ID NO: 2 and/or the 3′ UTR comprises the sequence of SEQ ID NO: 3. In some aspects, the functional OTC comprises the amino acid sequence of SEQ ID NO:7. In some aspects, the ORF sequence comprises SEQ ID NO: 1.

In some aspects, the mRNA sequence has no more than four, three, two, or one nucleic acids different from SEQ ID NO: 4. In certain aspects, the polynucleotide construct comprises the sequence of SEQ ID NO: 4.

In some aspects, the polynucleotide construct further comprises a 5′ terminal cap, e.g., Cap1. In some aspects, the polynucleotide construct further comprises a polyA tail. In certain aspects, the polyA tail is between 80 and 1000 nucleic acids long, e.g., between 100 and 500 nucleic acids long.

In some aspects, the mRNA comprises at least one chemically modified uridine.

In certain aspects, at least about 50%, at least about 60%, at least about 70%, at least about 80%, at least about 85%, at least about 90%, at least about 95%, or about 100% of the uridines are chemically modified. In some aspects, the chemically modified uridine is selected from the group consisting of pseudouridine (ψ), N1-methyl pseudouridine (N1-me-ψ), and/or a combination thereof.

Certain aspects of the disclosure are directed to a composition comprising: a polynucleotide construct of the disclosure; and a delivery agent. In some aspects, the delivery agent comprises a lipid nanoparticle (LNP), a liposome, a polymer, a micelle, a plasmid, a virus, or any combination thereof.

In certain aspects, the LNP is selected from the group consisting of compositions within LNP1 (PEG2000-C-DMA:13-B43:Cholesterol:DSPC), LNP2 (PEG2000-S:13-B43:Cholesterol:DSPC or PEG2000-S:18-B6:Cholesterol:DSPC), and LNP3 (PEG750-C-DLA:18-B6:Cholesterol:DSPC) groups. In some aspects, the polynucleotide construct is encapsulated in the LNP. In some aspects, the composition further comprises a pharmaceutically acceptable carrier. In some aspects, the polynucleotide construct is fully encapsulated in the LNP. In some aspects, at least 91%, at least 92%, at least 93%, at least 94%, at least 95%, at least 96%, at least 97%, at least 98%, at least 99%, or more of the polynucleotide construct is encapsulated by the LNP.

Certain aspects of the disclosure are directed to a method for increasing the amount of OTC expression in a cell comprising administering to the cell a composition comprising a polynucleotide construct of the disclosure or the composition of the disclosure. In some aspects, the cell is a liver cell.

Certain aspects of the disclosure are directed to a method for treating or reducing the symptoms associated with ornithine transcarbamylase deficiency (OTCD) comprising administering to a subject in need thereof a therapeutically effective amount of a composition comprising the polynucleotide construct of the disclosure or the composition of the disclosure.

Certain aspects of the disclosure are directed to a method for treating or reducing the risk of hyperammonemia in a subject with OTCD comprising administering to a subject in need thereof a therapeutically effective amount of a composition comprising the polynucleotide construct of the disclosure or the composition of the disclosure.

Certain aspects of the disclosure are directed to an expression cassette comprising a DNA sequence having at least 85%, at least 90%, at least 95%, at least 96%, at least 97%, at least 98%, at least 99%, or 100% sequence identity to SEQ ID NO: 8. In some aspects, the expression cassette further comprises a promoter, e.g., a T7 promoter. Some aspects of the disclosure are directed to a plasmid comprising the expression cassette of the disclosure. In some aspects, the expression cassette transcribes an mRNA of the disclosure (e.g., comprising SEQ ID NO: 1 or SEQ ID NO: 4). Some aspects of the disclosure are directed to a host cell comprising an expression cassette of the disclosure, or the plasmid of the disclosure.

Certain aspects of the disclosure are directed to use of the polynucleotide construct of the disclosure, or the composition of the disclosure, or the expression cassette of the disclosure, or the plasmid of the disclosure, or the host cell of the disclosure, for the manufacture of a medicament for the treatment of OTCD in a subject in need thereof or for the treatment of or for reducing the risk of hyperammonemia in a subject with OTCD.

Certain aspects of the disclosure are directed to methods for the in vivo delivery of a nucleic acid, the method comprising: administering to a mammalian subject a polynucleotide construct of the disclosure, or a composition of the disclosure, or an expression cassette of the disclosure, or a plasmid of the disclosure, or a host cell of the disclosure.

Certain aspects of the disclosure are directed to methods for treating a disease or disorder in a mammalian subject in need thereof, the method comprising: administering to the mammalian subject a therapeutically effective amount of a polynucleotide construct of the disclosure, or a composition of the disclosure, or an expression cassette of the disclosure, or a plasmid of the disclosure, or a host cell of the disclosure. In some aspects, the disease or disorder is a urea cycle disorder.

These and other aspects will be apparent from a reading of the following detailed description.

### BRIEF DESCRIPTION OF THE FIGURES

In some instances, the disclosure can be more completely understood in consideration of the following detailed description of various aspects of the disclosure in connection with the accompanying Figures, in which:

FIG. 1 shows MCP-1 induction at 6 hours after the first dose in rats administered LNP encapsulating codon optimized OTC constructs (OTC mRNA) having different poly(A) tail lengths (80, 161, 208, 262, 322, or 440 nucleotides) compared to PBS control. The 80 nucleotide poly(A) was encoded and the other tested poly(A) were enzymatic (enz).

FIG. 2A shows MCP-1 induction at 6 hours after the first, second, and third dose on Day 0, 7, and 14 respectively, in rats administered LNP encapsulating codon optimized OTC constructs (OTC mRNA) having different poly(A) tail lengths (80, 161, 208, 262, 322, or 440 nucleotides) compared to PBS control. The 80 nucleotide poly(A) was encoded and the other tested poly(A) were enzymatic (enz).

FIG. 2B shows IP-1 induction at 6 hours after the first, second, and third dose on Day 0, 7 and 14 respectively, in rats administered LNP encapsulating codon optimized OTC constructs (OTC mRNA) having different poly(A) tail lengths (80, 161, 208, 262, 322, or 440 nucleotides) compared to PBS control. The 80 nucleotide poly(A) was encoded and the other tested poly(A) were enzymatic (enz).

FIG. 3A shows hOTC protein expression in rat livers after a single dose administration of LNP encapsulating codon optimized OTC constructs (OTC mRNA) having different poly(A) tail lengths (80, 161, 208, 262, 322, or 440 nucleotides) compared to PBS control. The 80 nucleotide poly(A) was encoded and the other tested poly(A) were enzymatic (enz).

FIG. 3B shows hOTC protein expression in rat livers after a single versus multi-dose administration of LNP carrying codon optimized OTC constructs (OTC mRNA) having different poly(A) tail lengths (80, 161, 208, 262, 322, or 440 nucleotides) compared to PBS control. The 80 nucleotide poly(A) was encoded and the other tested poly(A) were enzymatic (enz).

FIG. 4 shows MCP-1 induction at 6 hours after the first dose in mice administered with LNP1 or LNP2 (ionizable lipid: 13-B43) groups encapsulating OTC constructs (OTC mRNA) with different modifications: PsU, N1MePsU, or 5MoU, compared to PBS control.

FIG. 5 shows hOTC expression at 24 hours post dose in mice administered with LNP1 or LNP2 (ionizable lipid: 13-B43) groups encapsulating OTC constructs with different modifications: PsU, N1MePsU, or SMoU, compared to PBS control.

FIG. 6A shows anti-PEG IgG antibody response in rats administered different LNP (LNP1, LNP2 (ionizable lipid: 13-B43), LNP2 (ionizable lipid: 18-B6), or LNP3) groups encapsulating a codon optimized OTC construct (OTC mRNA) compared to EPO and Luc payloads.

FIG. 6B shows anti-PEG IgM antibody response in rats administered different LNP (LNP1, LNP2 (ionizable lipid: 13-B43), LNP2 (ionizable lipid: 18-B6), or LNP3) groups encapsulating a codon optimized OTC construct (OTC mRNA) compared to EPO and Luc payloads.

FIG. 7 shows MCP-1 induction at 6 hours in rats administered different LNP (LNP1, LNP2 (ionizable lipid: 13-B43), LNP2 (ionizable lipid: 18-B6), or LNP3) groups encapsulating a codon optimized OTC construct (OTC mRNA) compared to EPO and Luc payloads and PBS at 0, 7 and 14 days.

FIG. 8 shows OTC protein expression in rats administered different LNP (LNP1, LNP2 (ionizable lipid: 13-B43), LNP2 (ionizable lipid: 18-B6), or LNP3) groups encapsulating a codon optimized OTC constructs (OTC mRNA) after 1 and 3 doses.

FIG. 9 shows lipid concentration (clearance) in rat livers following administered different LNP (LNP1, LNP2 (ionizable lipid: 13-B43), LNP2 (ionizable lipid: 18-B6), or LNP3) groups encapsulating a codon optimized OTC constructs (OTC mRNA) after 1 and 3 doses.

FIG. 10A shows ALT levels in rats following administered different LNP (LNP1, LNP2 (ionizable lipid: 13-B43), LNP2 (ionizable lipid: 18-B6), or LNP3) groups encapsulating a codon optimized OTC constructs (OTC mRNA) after 1 and 3 doses.

FIG. 10B shows AST levels in rats following administered different LNP (LNP1, LNP2 (ionizable lipid: 13-B43), LNP2 (ionizable lipid: 18-B6), or LNP3) groups encapsulating a codon optimized OTC constructs (OTC mRNA) after 1 and 3 doses.

FIG. 11A-11C shows cytokine response following administration of an LNP2 (ionizable lipid: 13-B43) composition encapsulating a codon optimized OTC constructs after weekly repeat doses. FIG. 11A shows MCP-1 induction 6 hours post dose, FIG. 11B shows IP-10 induction 6 hours post dose, and FIG. 11C shows MIP-1a induction 6 hours post dose.

FIG. 12 shows anti-PEG IgM antibody response following administration of LNP2 (ionizable lipid: 13-B43) encapsulating a codon optimized OTC constructs after weekly repeat doses compared to PBS control.

FIG. 13 shows anti-PEG IgG antibody response following administration of an LNP2 (ionizable lipid: 13-B43) composition encapsulating a codon optimized OTC constructs after weekly repeat doses compared to PBS control.

FIG. 14 shows anti-OTC IgM antibody response following administration of an LNP2 (ionizable lipid: 13-B43) composition encapsulating a codon optimized OTC construct after weekly repeat doses compared to PBS control.

FIG. 15 shows anti-OTC IgM antibody response following administration of an LNP2 (ionizable lipid: 13-B43) composition encapsulating a codon optimized OTC construct after weekly repeat doses compared to PBS control.

FIG. 16 shows OTC protein expression in rats administered an LNP2 (ionizable lipid: 13-B43) composition encapsulating a codon optimized OTC construct after weekly repeat doses.

FIG. 17A-17B show human OTC mRNA (hOTC mRNA) in (A) liver and (B) plasma of rats administered an LNP2 (ionizable lipid: 13-B43) composition encapsulating a codon optimized OTC construct.

FIG. 18A shows the average ALT levels 24 hours post-dose in the liver of rats administered an LNP1, LNP2 (ionizable lipid: 13-B43) or LNP2 (ionizable lipid: 18-B6) composition encapsulating a codon optimized OTC construct.

FIG. 18B shows the average AST levels 24 hours post-dose in the liver of rats administered an LNP1, LNP2 (ionizable lipid: 13-B43) or LNP2 (ionizable lipid: 18-B6) composition encapsulating a codon optimized OTC construct.

FIG. 18C shows the individual (R1, R2, or R3) and average ALT levels 24 hours post-dose in the liver of rats administered an LNP1, LNP2 (ionizable lipid: 13-B43) or LNP2 (ionizable lipid: 18-B6) composition encapsulating a codon optimized OTC construct.

FIG. 18D shows the individual (R1, R2, or R3) and average AST1 levels 24 h post-dose in the liver of rats administered an LNP1, LNP2 (ionizable lipid: 13-B43) or LNP2 (ionizable lipid: 18-B6) composition encapsulating a codon optimized OTC construct.

FIGS. 19A-19D shows (A) the average GGT levels, (B) total bilirubin levels, (C) individual (R1, R2, or R3) and average GGT levels, and (D) individual (R1, R2, or R3) and average total bilirubin levels 24 hours post-dose of rats administered an LNP1, LNP2 (ionizable lipid: 13-B43) or LNP2 (ionizable lipid: 18-B6) composition encapsulating a codon optimized OTC construct.

FIG. 20A-20C shows (A) the neutrophil levels, (B) the monocyte levels, and (C) the platelet levels at 24 hours post-dose of rats administered an LNP1, LNP2 (ionizable lipid: 13-B43) or LNP2 (ionizable lipid: 18-B6) composition encapsulating a codon optimized OTC construct.

FIG. 21A-21C shows (A) the MCP-1 levels, (B) the MIP-1a levels, and (C) the IP-10 levels at 6 hours post-dose of rats administered an LNP1, LNP2 (ionizable lipid: 13-B43) or LNP2 (ionizable lipid: 18-B6) composition encapsulating a codon optimized OTC construct.

FIG. 22 shows OTC expression at 24 hours post-dose of rats administered an LNP1 or LNP2 (ionizable lipid: 13-B43) composition encapsulating a codon optimized OTC construct.

FIGS. 23A-23C show (A) human OTC (hOTC), (B) MCP-1, and (C) IL-6 protein expression levels in the livers of non-human primates that were administered LNP1 encapsulating a codon optimized OTC mRNA construct at 0.25 mg/kg, 1 mg/kg, and 3 mg/kg. The hOTC protein expression is shown as % of endogenous, and the MCP-1 and IL-6 protein expression are shown compared to 0 mg/kg control.

### DETAILED DESCRIPTION

The present disclosure is directed to improved polynucleotides (e.g., mRNA), compositions, and methods for expressing functional enzyme ornithine transcarbamylase (OTC) in a cell and use of such polynucleotides, compositions, and methods for treating a subject suffering from OTC deficiency. Unless defined otherwise, all technical and scientific terms used herein have the same meaning as commonly understood by one of ordinary skill in the art pertinent to the methods and compositions described. The definitions provided herein are to facilitate understanding of certain terms used frequently herein.

As used in this specification and the appended claims, the singular forms “a”, “an”, and “the” encompass aspects having plural referents, unless the content clearly dictates otherwise.

As used herein, the term “nucleic acid,” in its broadest sense, refers to any compound and/or substance that is or can be incorporated into a polynucleotide chain, e.g., via a phosphodiester linkage. In some aspects, “nucleic acid” refers to individual nucleic acid residues (e.g., nucleotides and/or nucleosides). In some aspects, “nucleic acid” refers to a polynucleotide chain comprising individual nucleic acid residues. In some aspects, “nucleic acid” encompasses RNA, e.g., mRNA, as well as single and/or double-stranded DNA and/or cDNA.

As used herein the term “polynucleotide” or “oligonucleotide” refers to a polymer comprising 7-20,000 nucleotide monomeric units (i.e., from 7 nucleotide monomeric units to 20,000 nucleotide monomeric units, inclusive). Polynucleotides include deoxyribonucleic acid (DNA) and ribonucleic acid (RNA), or their derivatives, and combinations of DNA and RNA. For example, DNA can be in form of cDNA, in vitro polymerized DNA, plasmid DNA, parts of a plasmid DNA, expression vectors, expression cassettes, chimeric sequences, recombinant DNA, chromosomal DNA, or any derivatives thereof. In further examples, RNA can be in the form of messenger RNA (mRNA), in vitro polymerized RNA, recombinant RNA, transfer RNA (tRNA), small nuclear RNA (snRNA), ribosomal RNA (rRNA), chimeric sequences, recombinant RNA, or any derivatives thereof. In addition, DNA and RNA can be single, double, triple, or quadruple stranded.

Further examples of polynucleotides as used herein include, but are not limited to single stranded mRNA which can be modified or unmodified. Modified mRNA includes those with at least two modifications and a translatable region. The modifications can be located on the backbone and/or a nucleoside of the nucleic acid molecule. The modifications can be located on both a nucleoside and a backbone linkage.

As used herein, the term “messenger RNA (mRNA)” refers to a polyribonucleotide that encodes at least one polypeptide. mRNA as used herein encompasses both modified and unmodified RNA. mRNA can contain one or more coding and non-coding regions. mRNA can be purified from natural sources, produced using recombinant expression systems and optionally purified, in vitro transcribed, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. In some aspects, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

As used herein, “expression” of a nucleic acid sequence refers to translation of a polynucleotide, e.g., an mRNA, into a polypeptide, assembly of multiple polypeptides into an intact protein (e.g., enzyme) and/or post-translational modification of a polypeptide or fully assembled protein (e.g., enzyme). In this disclosure, the terms “expression” and “production,” and grammatical equivalent, are used inter-changeably.

As used herein, the term “amino acid,” in its broadest sense, refers to any compound and/or substance that can be incorporated into a polypeptide chain. In some aspects, an amino acid has the general structure H₂N—C(H)(R)—COOH. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids can participate in a disulfide bond. Amino acids can comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and can refer to a free amino acid and/or to an amino acid residue of a peptide. It will be apparent from the context in which the term is used whether it refers to a free amino acid or a residue of a peptide.

A “polypeptide” is a polymer of amino acid residues joined by peptide bonds, whether produced naturally or synthetically.

As used herein the term “peptide” refers to a polypeptide having 2-100 amino acid monomers.

A “protein” is a macromolecule comprising one or more polypeptide chains. A protein can also comprise non-peptidic components, such as carbohydrate groups. Carbohydrates and other non-peptidic substituents can be added to a protein by the cell in which the protein is produced, and will vary with the type of cell. Some proteins are defined herein in terms of their amino acid backbone structures.

As used herein, a “functional” biological molecule, e.g., a protein, is a biological molecule in a form in which it exhibits a property and/or activity by which it is characterized.

As used herein, the term “delivery” encompasses both local and systemic delivery. For example, delivery of a polynucleotide, e.g., an mRNA, encompasses situations in which a polynucleotide is delivered to a target tissue and the encoded protein is expressed and retained within the target tissue (also referred to as “local distribution” or “local delivery”). Other exemplary situations include one in which a polynucleotide is delivered to a target tissue and the encoded protein is expressed and secreted into patient's circulation system (e.g., serum) and systematically distributed and taken up by other tissues (also referred to as “systemic distribution” or “systemic delivery). In other exemplary situations, a polynucleotide is delivered systemically and is taken up in a wide variety of cells and tissues in vivo. In some exemplary situations, the delivery is intravenous, intramuscular or subcutaneous.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a human and a non-human animal. In the context of cell-based systems, the term can be used to refer to events that occur within a living cell (as opposed to, for example, in vitro systems).

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein the term “treating” refers to the administration of a delivery agent and nucleic acid that eliminates, alleviates, inhibits the progression of, or reverses progression of, in part or in whole, any one or more of the pathological hallmarks or symptoms of any one of the diseases and disorders being treated. Such diseases include, but are not limited to, ornithine transcarbamylase deficiency (OTCD).

The phrase “therapeutically effective” as used herein is intended to qualify the amount of polynucleotide or pharmaceutical composition, or the combined amount of active ingredients in the case of combination therapy. This amount or combined amount will achieve the goal of treating the relevant disease or condition.

As used herein, the term “subject” refers to a human or any non-human animal (e.g., mouse, rat, rabbit, dog, cat, cattle, swine, sheep, horse or primate). A human includes pre- and post-natal forms. In many aspects, a subject is a human. A subject can be a patient, which refers to a human presenting to a medical provider for diagnosis or treatment of a disease. The term “subject” can be used herein interchangeably with “individual” or “patient.” A subject can be afflicted with or is susceptible to a disease or disorder but may or may not display symptoms of the disease or disorder.

The term “lipid” refers to a group of organic compounds that are esters of fatty acids and are characterized by being insoluble in water but soluble in many organic solvents. They are usually divided in at least three classes: (1) “simple lipids” which include fats and oils as well as waxes; (2) “compound lipids” which include phospholipids and glycolipids; (3) “derived lipids” such as steroids.

The term “amphipathic lipid” refers, in part, to any suitable material wherein the hydrophobic portion of the lipid material orients into a hydrophobic phase, while a hydrophilic portion orients toward the aqueous phase. Amphipathic lipids are usually the major component of a lipid LNP. Hydrophilic characteristics derive from the presence of polar or charged groups such as carbohydrates, phosphato, carboxylic, sulfato, amino, sulfhydryl, nitro, hydroxy and other like groups. Hydrophobicity can be conferred by the inclusion of apolar groups that include, but are not limited to, long chain saturated and unsaturated aliphatic hydrocarbon groups and such groups substituted by one or more aromatic, cycloaliphatic or heterocyclic group(s). Examples of amphipathic compounds include, but are not limited to, phospholipids, aminolipids and sphingolipids. Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, di stearoylphosphatidylcholine or dilinoleoylphosphatidylcholine. Other compounds lacking in phosphorus, such as sphingolipid, glycosphingolipid families, diacylglycerols and β-acyloxyacids, are also within the group designated as amphipathic lipids. Additionally, the amphipathic lipid described above can be mixed with other lipids including triglycerides and sterols.

The term “anionic lipid” refers to any lipid that is negatively charged at physiological pH. These lipids include, but are not limited to, phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, and other anionic modifying groups joined to neutral lipids.

The term “cationic lipid” refers to any of a number of lipid species which carry a net positive charge at a selective pH, such as physiological pH. Such lipids include, but are not limited to, N,N-dioleyl-N,N-dimethylammonium chloride (“DODAC”); N-(2,3-dioleyloxy)propyl)-N,N,N-trimethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 3-(N—(N′,N′-dimethylaminoethane)-carbamoyl)cholesterol (“DC-Chol”) and N-(1,2-dimyristyloxyprop-3-yl)-N,N-dimethyl-N-hydroxyethyl ammonium bromide (“DMRIE”). Additionally, a number of commercial preparations of cationic lipids are available which can be used in the present disclosure. These include, for example, LIPOFECTIN® (commercially available cationic liposomes comprising DOTMA and 1,2-dioleoyl-sn-3-phosphoethanolamine (“DOPE”), from GIBCO/BRL, Grand Island, N.Y., USA); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (“DOSPA”) and (“DOPE”), from GIBCO/BRL); and TRANSFECTAM® (commercially available cationic lipids comprising dioctadecylamidoglycyl carboxyspermine (“DOGS”) in ethanol from Promega Corp., Madison, Wis., USA). The following lipids are cationic and have a positive charge at below physiological pH: DODAP, DODMA, DMDMA and the like.

The term “lipid nanoparticle” refers to any lipid composition that can be used to deliver a compound (e.g., a polynucleotide construct) including, but not limited to, liposomes, wherein an aqueous volume is encapsulated by an amphipathic lipid bilayer; or wherein the lipids coat an interior comprising a large molecular component, such as a plasmid, with a reduced aqueous interior; or lipid aggregates or micelles, wherein the encapsulated component is contained within a relatively disordered lipid mixture.

As used herein, “lipid encapsulated” or “lipid encapsulation” can refer to a lipid formulation which provides a compound (e.g., a polynucleotide construct) with full encapsulation, partial encapsulation, or both. “Full encapsulation” or “fully encapsulated” is understood herein to mean at least 90% a compound (e.g., a polynucleotide construct) in a lipid formulation is encapsulated by the lipid (e.g., LNP). In some aspects, at least 95%, at least 96%, at least 97%, at least 98%, at least 99%, or more of the compound (e.g., a polynucleotide construct) in a lipid formulation is encapsulated by the lipid (e.g., LNP).

### Polynucleotide Constructs

The polynucleotide constructs disclosed herein can be used as therapeutic agents to increase the level of an OTC protein in a cell (in vitro or in vivo) to a level greater than that obtained and/or observed in the absence of the polynucleotide constructs disclosed herein.

In certain aspects, the polynucleotide construct comprises a nucleic acid sequence, e.g., an mRNA sequence, comprising an open reading frame (ORF) encoding a functional human ornithine transcarbamylase (OTC) protein. The ORF can encode a full length OTC protein or a functional fragment thereof. In some aspects, the ORF encodes an amino acid sequence having at least 85%, at least 90%, at least 91%, at least 92%, at least 93%, at least 94%, at least 95%, at least 96%, at least 97%, at least 98%, at least 99%, at least 99.5%, or 100% sequence identity to SEQ ID NO: 7. In some aspects, the full length OTC comprises the amino acid sequence of SEQ ID NO: 7.

In some aspects, the polynucleotide construct comprises an mRNA sequence comprising an ORF which is codon optimized. In some aspects, the ORF comprises a sequence having at least 95%, at least 96%, at least 97%, at least 98%, at least 99%, at least 99.5%, or 100% sequence identity to SEQ ID NO: 1. In some aspects, the ORF comprises the nucleic acid sequence of SEQ ID NO: 1.

In some aspects, the polynucleotide construct comprises a 5′ UTR. The 5′ UTR can comprise the sequence of SEQ ID NO: 2.

In some aspects, the polynucleotide construct comprises a 3′ UTR. The 3′ UTR can comprise the sequence of SEQ ID NO: 3

In some aspects, a polynucleotide construct of the disclosure comprises, from 5′ to 3′: (i) a 5′ UTR, e.g., comprising the sequence of SEQ ID NO: 2; (ii) a nucleic acid sequence, e.g., a mRNA, comprising an open reading frame (ORF) encoding a functional human ornithine transcarbamylase (OTC), wherein ORF comprises a sequence at least about 95%, 96%, 97%, 98%, 99%, 99.5%, or 100% identical to SEQ ID NO: 1; and a 3′ UTR comprising the sequence of SEQ ID NO: 3.

In some aspects, the polynucleotide construct comprises a sequence no more than five nucleic acids different from SEQ ID NO: 4. In some aspects, the polynucleotide construct comprises a sequence having five, four, three, two, or one nucleotide differences from SEQ ID NO: 4. In some aspects, the nucleic acid differences can be present within nucleotides 2 to 1221 of SEQ ID NO: 4. The polynucleotide construct can comprise the sequence of SEQ ID NO: 4.

The polynucleotide construct can further comprise a polyA tail. In some aspects, the polyA tail is longer than 80, 81, 82, 83, 84, 85, 86, 87, 88, 89, 90, 91, 92, 93, 94, 95, 96, 97, 98, 99, 100, 110, 115, 120, 125, 130, 135, 140, 145, or 150 nucleic acids. In some aspects, the polyA tail is between 80 to 1000, 85 to 1000, 90 to 1000, 95 to 1000, 100 to 1000, 105 to 1000, 110 to 1000, 115 to 1000, 120 to 1000, 125 to 1000, 130 to 1000, 135 to 1000, 140 to 1000, 145 to 1000, 150 to 1000, 155 to 1000, 160 to 1000, 80 to 800, 85 to 800, 90 to 800, 95 to 800, 100 to 800, 105 to 800, 110 to 800, 115 to 800, 120 to 800, 125 to 800, 130 to 800, 135 to 800, 140 to 800, 145 to 800, 150 to 800, 155 to 800, or 160 to 800 nucleic acids long. In some aspects, the polyA tail is between 100 and 500 nucleic acids long.

In some aspects, the polynucleotide construct comprises a start codon at the 5′ end of the ORF. In some aspects, the polynucleotide construct comprises a stop codon at the 3′ end of the ORF.

In certain aspects, the polynucleotide construct comprises a modified nucleotide. In some aspects, the polynucleotide construct comprises an mRNA sequence comprising an open reading frame (ORF) encoding a functional human ornithine transcarbamylase (OTC), wherein the mRNA sequence comprises a modified nucleotide. In some aspects, the modified nucleotide is uridine. In some aspects, at least about 50%, at least about 60%, at least about 70%, at least about 80%, at least about 90%, at least about 95%, at least about 99%, or 100% of the uridines are chemically modified.

In some aspects, the chemically modified uridine is selected from the group consisting of pseudouridine (ψ), N1-methyl pseudouridine (N1-me-ψ), 5-methoxy uridine (5moU), and any combination thereof. In some aspects, the chemically modified uridine is selected from the group consisting of pseudouridine (ψ), N1-methyl pseudouridine (N1-me-ψ), and any combination thereof. In certain aspects, the ORF, e.g., comprising SEQ ID NO: 1, comprises at least 95%, at least 98%, at least 99%, or about 100% modified uridines, e.g., pseudouridine (ψ) modified modified or N1-methyl pseudouridine (N1-me-ψ) modified.

In certain aspects, the polynucleotide construct can be prepared using an expression cassette comprising a sequence having at least 85%, at least 90%, at least 95%, at least 96%, at least 97%, at least 98%, at least 99%, or 100% sequence identity to SEQ ID NO: 8. In some aspects, the expression cassette further comprises a promoter, e.g., a T7 promoter. In some aspects, the T7 promoter comprises the following 5′ to 3′ sequence: TAATACGACTCACTATA (SEQ ID NO: 9). In some aspects, the 5′ UTR of the expression cassette comprises an adenine (A) immediately downstream of the promoter, e.g., T7 promoter. Some aspects are directed to a plasmid comprising the expression cassette. In some aspects, the plasmid further comprises an antibiotic resistance gene. In some aspects, the polynucleotide construct is prepared using in vitro transcription.

Exemplary OTC amino acid sequences and encoding nucleotide sequences are shown in Table 1 herein.

TABLE 1
Sequence Related to Polynucleotide Constructs

| SEQ ID | | |
|---|---|---|
| NO: | Sequence | Description |
| 1 | 5′- | human OTC |
| | AUGCUGUUUAACCUGAGGAUUCUGCUGAACAACGCUGCUUUU | mRNA-coding |
| | CGGAACGGCCACAACUUUAUGGUGCGGAACUUUCGGUGCGGA | sequence |
| | CAGCCACUGCAGAACAAAGUGCAGCUGAAGGGGAGGGACCUG | |
| | CUGACCCUGAAAAAUUUCACAGGAGAGGAAAUCAAGUACAUG | |
| | CUGUGGCUGUCUGCCGAUCUGAAGUUCCGGAUCAAGCAGAAG | |
| | GGCGAAUAUCUGCCACUGCUGCAGGGCAAAAGUCUGGGGAUG | |
| | AUCUUCGAAAAGAGGAGUACUCGGACCAGACUGUCAACAGAG | |
| | ACUGGAUUCGCUCUGCUGGGAGGACACCCAUGCUUUCUGACC | |
| | ACACAGGACAUUCAUCUGGGCGUGAACGAGUCACUGACCGAC | |
| | ACAGCUCGAGUCCUGAGCUCCAUGGCAGAUGCCGUGCUGGCA | |
| | CGGGUCUACAAACAGAGCGACCUGGAUACCCUGGCUAAGGAA | |
| | GCAAGCAUCCCCAUCAUUAAUGGGCUGUCCGACCUGUAUCAC | |
| | CCUAUCCAGAUUCUGGCCGAUUACCUGACCCUGCAGGAGCAU | |
| | UAUUCUAGUCUGAAAGGCCUGACACUGAGCUGGAUUGGGGAC | |
| | GGAAACAAUAUCCUGCACUCCAUUAUGAUGUCUGCCGCUAAG | |
| | UUUGGAAUGCAUCUGCAGGCAGCCACACCAAAAGGCUACGAA | |
| | CCCGAUGCCAGUGUGACUAAGCUGGCCGAACAGUAUGCUAAA | |
| | GAGAACGGCACUAAGCUGCUGCUGACCAAUGACCCUCUGGAG | |
| | GCUGCACACGGAGGCAACGUCCUGAUCACUGAUACCUGGAUU | |
| | UCCAUGGGCCAGGAGGAAGAGAAGAAAAAGCGCCUGCAGGCA | |
| | UUCCAGGGGUACCAGGUGACAAUGAAAACUGCCAAGGUCGCC | |
| | GCUUCUGAUUGGACUUUUCUGCAUUGUCUGCCCCGAAAACCU | |
| | GAAGAGGUGGACGAUGAGGUCUUCUAUUCACCUAGGAGCCUG | |
| | GUGUUUCCAGAAGCCGAGAAUCGCAAGUGGACAAUCAUGGCU | |
| | GUGAUGGUGUCCCUGCUGACUGAUUAUUCCCCCCAGCUGCAG | |
| | AAACCUAAGUUCUGA-3′ | |
| 2 | AGGAAAUAAGAGAGAAAAGAAGAGUAAGAAGAAAUAUAAGAG | 5′ UTR |
| | CCACC | |
| 3 | GCGGCCGCUUAAUUAAGCUGCCUUCUGCGGGGCUUGCCUUCU | 3′ UTR |
| | GGCCAUGCCCUUCUUCUCUCCCUUGCACCUGUACCUCUUGGU | |
| | CUUUGAAUAAAGCCUGAGUAGGAAGUCUAG | |
| 4 | 5′- | 5′ UTR, human |
| | AGGAAAUAAGAGAGAAAAGAAGAGUAAGAAGAAAUAUAAGAG | OTC mRNA ORF, |
| | CCACCAUGCUGUUUAACCUGAGGAUUCUGCUGAACAACGCUG | 3′ UTR Sequence: |
| | CUUUUCGGAACGGCCACAACUUUAUGGUGCGGAACUUUCGGU | start and stop |
| | GCGGACAGCCACUGCAGAACAAAGUGCAGCUGAAGGGGAGGG | codons are |
| | ACCUGCUGACCCUGAAAAAUUUCACAGGAGAGGAAAUCAAGU | underlined |
| | ACAUGCUGUGGCUGUCUGCCGAUCUGAAGUUCCGGAUCAAGC | |
| | AGAAGGGCGAAUAUCUGCCACUGCUGCAGGGCAAAAGUCUGG | |
| | GGAUGAUCUUCGAAAAGAGGAGUACUCGGACCAGACUGUCAA | |
| | CAGAGACUGGAUUCGCUCUGCUGGGAGGACACCCAUGCUUUC | |
| | UGACCACACAGGACAUUCAUCUGGGCGUGAACGAGUCACUGA | |
| | CCGACACAGCUCGAGUCCUGAGCUCCAUGGCAGAUGCCGUGC | |
| | UGGCACGGGUCUACAAACAGAGCGACCUGGAUACCCUGGCUA | |
| | AGGAAGCAAGCAUCCCCAUCAUUAAUGGGCUGUCCGACCUGU | |
| | AUCACCCUAUCCAGAUUCUGGCCGAUUACCUGACCCUGCAGG | |
| | AGCAUUAUUCUAGUCUGAAAGGCCUGACACUGAGCUGGAUUG | |
| | GGGACGGAAACAAUAUCCUGCACUCCAUUAUGAUGUCUGCCG | |
| | CUAAGUUUGGAAUGCAUCUGCAGGCAGCCACACCAAAAGGCU | |
| | ACGAACCCGAUGCCAGUGUGACUAAGCUGGCCGAACAGUAUG | |
| | CUAAAGAGAACGGCACUAAGCUGCUGCUGACCAAUGACCCUC | |
| | UGGAGGCUGCACACGGAGGCAACGUCCUGAUCACUGAUACCU | |
| | GGAUUUCCAUGGGCCAGGAGGAAGAGAAGAAAAAGCGCCUGC | |
| | AGGCAUUCCAGGGGUACCAGGUGACAAUGAAAACUGCCAAGG | |
| | UCGCCGCUUCUGAUUGGACUUUUCUGCAUUGUCUGCCCCGAA | |
| | AACCUGAAGAGGUGGACGAUGAGGUCUUCUAUUCACCUAGGA | |
| | GCCUGGUGUUUCCAGAAGCCGAGAAUCGCAAGUGGACAAUCA | |
| | UGGCUGUGAUGGUGUCCCUGCUGACUGAUUAUUCCCCCCAGC | |
| | UGCAGAAACCUAAGUUCUGAGCGGCCGCUUAAUUAAGCUGCC | |
| | UUCUGCGGGGCUUGCCUUCUGGCCAUGCCCUUCUUCUCUCCC | |
| | UUGCACCUGUACCUCUUGGUCUUUGAAUAAAGCCUGAGUAGG | |
| | AAGUCUAG-3′ | |
| 6 | 5′- | Exemplary |
| | AGGAAAUAAGAGAGAAAAGAAGAGUAAGAAGAAAUAUAAGAG | polynucleotide |
| | CCACCAUGCUGUUUAACCUGAGGAUUCUGCUGAACAACGCUG | construct with start |
| | CUUUUCGGAACGGCCACAACUUUAUGGUGCGGAACUUUCGGU | and stop codon |
| | GCGGACAGCCACUGCAGAACAAAGUGCAGCUGAAGGGGAGGG | underlined |
| | ACCUGCUGACCCUGAAAAAUUUCACAGGAGAGGAAAUCAAGU | |
| | ACAUGCUGUGGCUGUCUGCCGAUCUGAAGUUCCGGAUCAAGC | |
| | AGAAGGGCGAAUAUCUGCCACUGCUGCAGGGCAAAAGUCUGG | |
| | GGAUGAUCUUCGAAAAGAGGAGUACUCGGACCAGACUGUCAA | |
| | CAGAGACUGGAUUCGCUCUGCUGGGAGGACACCCAUGCUUUC | |
| | UGACCACACAGGACAUUCAUCUGGGCGUGAACGAGUCACUGA | |
| | CCGACACAGCUCGAGUCCUGAGCUCCAUGGCAGAUGCCGUGC | |
| | UGGCACGGGUCUACAAACAGAGCGACCUGGAUACCCUGGCUA | |
| | AGGAAGCAAGCAUCCCCAUCAUUAAUGGGCUGUCCGACCUGU | |
| | AUCACCCUAUCCAGAUUCUGGCCGAUUACCUGACCCUGCAGG | |
| | AGCAUUAUUCUAGUCUGAAAGGCCUGACACUGAGCUGGAUUG | |
| | GGGACGGAAACAAUAUCCUGCACUCCAUUAUGAUGUCUGCCG | |
| | CUAAGUUUGGAAUGCAUCUGCAGGCAGCCACACCAAAAGGCU | |
| | ACGAACCCGAUGCCAGUGUGACUAAGCUGGCCGAACAGUAUG | |
| | CUAAAGAGAACGGCACUAAGCUGCUGCUGACCAAUGACCCUC | |
| | UGGAGGCUGCACACGGAGGCAACGUCCUGAUCACUGAUACCU | |
| | GGAUUUCCAUGGGCCAGGAGGAAGAGAAGAAAAAGCGCCUGC | |
| | AGGCAUUCCAGGGGUACCAGGUGACAAUGAAAACUGCCAAGG | |
| | UCGCCGCUUCUGAUUGGACUUUUCUGCAUUGUCUGCCCCGAA | |
| | AACCUGAAGAGGUGGACGAUGAGGUCUUCUAUUCACCUAGGA | |
| | GCCUGGUGUUUCCAGAAGCCGAGAAUCGCAAGUGGACAAUCA | |
| | UGGCUGUGAUGGUGUCCCUGCUGACUGAUUAUUCCCCCCAGO | |
| | UGCAGAAACCUAAGUUCUGAGCGGCCGCUUAAUUAAGCUGCC | |
| | UUCUGCGGGGCUUGCCUUCUGGCCAUGCCCUUCUUCUCUCCC | |
| | UUGCACCUGUACCUCUUGGUCUUUGAAUAAAGCCUGAGUAGG | |
| | AAGUCUAGAAAAAAAAAAAAAAAAAAAAAAAAAAAAAAAAAA | |
| | AAAAAAAAAAAAAAAAAAAAAAAAAAAAAAAAAAAAAAAAAA | |
| | AAAAAAAAAAAAAAAAAAAAAAAAAAAAAAAAAAAAAAAAAA | |
| | AAAAAAAAAAAAAAAAAAAAAAAAAAAAAAAAAAAAAAAAAA | |
| | AAAAAAAAAAAAAAAAAAAAAAAAAAAAAAAAAAAAAAAAAA | |
| | AAAAAAAAAAAAAAAAAAAAAAAAAAAAAAAAAAAAAAAAAA | |
| | AAAAAAAAAAAAAAAAAAAAAAAAAAAAAAAAAAAAAAAAAA | |
| | AAAAAAAAAAAAAAAAAAAAAAAAAAAAAAAAAAAAAAAAAA | |
| | AAAAAAAAAAAAAAAAAAAAAAAAAAAAAAAAAAAAAAAAAA | |
| | AAAAAAAAAAAAAAAAAAAAAAAAAAAAAAAAAAAAAAAAAA | |
| | AAAAAAAAAAAAAAAAAAAAAAAAAAAA-3′ | |
| 7 | MLFNLRILLNNAAFRNGHNFMVRNFRCGQPLQNKVQLKGRDL | Human OTC |
| | LTLKNFTGEEIKYMLWLSADLKFRIKQKGEYLPLLQGKSLGM | sequence (with |
| | IFEKRSTRTRLSTETGFALLGGHPCFLTTQDIHLGVNESLTD | native (human) |
| | TARVLSSMADAVLARVYKQSDLDTLAKEASIPIINGLSDLYH | mitochondrial |
| | PIQILADYLTLQEHYSSLKGLTLSWIGDGNNILHSIMMSAAK | leader sequence |
| | FGMHLQAATPKGYEPDASVTKLAEQYAKENGTKLLLTNDPLE | underlined) |
| | AAHGGNVLITDTWISMGQEEEKKKRLQAFQGYQVTMKTAKVA | amino acid |
| | ASDWTFLHCLPRKPEEVDDEVFYSPRSLVFPEAENRKWTIMA | |
| | VMVSLLTDYSPQLQKPKF | |
| 8 | AGGAAATAAGAGAGAAAAGAAGAGTAAGAAGAAATATAAGAG | DNA sequence for |
| | CCACCATGCTGTTTAACCTGAGGATTCTGCTGAACAACGCTG | 5′ UTR, human |
| | CTTTTCGGAACGGCCACAACTTTATGGTGCGGAACTTTCGGT | OTC open reading |
| | GCGGACAGCCACTGCAGAACAAAGTGCAGCTGAAGGGGAGGG | frame and 3′ UTR |
| | ACCTGCTGACCCTGAAAAATTTCACAGGAGAGGAAATCAAGT | |
| | ACATGCTGTGGCTGTCTGCCGATCTGAAGTTCCGGATCAAGC | |
| | AGAAGGGCGAATATCTGCCACTGCTGCAGGGCAAAAGTCTGG | |
| | GGATGATCTTCGAAAAGAGGAGTACTCGGACCAGACTGTCAA | |
| | CAGAGACTGGATTCGCTCTGCTGGGAGGACACCCATGCTTTC | |
| | TGACCACACAGGACATTCATCTGGGCGTGAACGAGTCACTGA | |
| | CCGACACAGCTCGAGTCCTGAGCTCCATGGCAGATGCCGTGC | |
| | TGGCACGGGTCTACAAACAGAGCGACCTGGATACCCTGGCTA | |
| | AGGAAGCAAGCATCCCCATCATTAATGGGCTGTCCGACCTGT | |
| | ATCACCCTATCCAGATTCTGGCCGATTACCTGACCCTGCAGG | |
| | AGCATTATTCTAGTCTGAAAGGCCTGACACTGAGCTGGATTG | |
| | GGGACGGAAACAATATCCTGCACTCCATTATGATGTCTGCCG | |
| | CTAAGTTTGGAATGCATCTGCAGGCAGCCACACCAAAAGGCT | |
| | ACGAACCCGATGCCAGTGTGACTAAGCTGGCCGAACAGTATG | |
| | CTAAAGAGAACGGCACTAAGCTGCTGCTGACCAATGACCCTC | |
| | TGGAGGCTGCACACGGAGGCAACGTCCTGATCACTGATACCT | |
| | GGATTTCCATGGGCCAGGAGGAAGAGAAGAAAAAGCGCCTGC | |
| | AGGCATTCCAGGGGTACCAGGTGACAATGAAAACTGCCAAGG | |
| | TCGCCGCTTCTGATTGGACTTTTCTGCATTGTCTGCCCCGAA | |
| | AACCTGAAGAGGTGGACGATGAGGTCTTCTATTCACCTAGGA | |
| | GCCTGGTGTTTCCAGAAGCCGAGAATCGCAAGTGGACAATCA | |
| | TGGCTGTGATGGTGTCCCTGCTGACTGATTATTCCCCCCAGC | |
| | TGCAGAAACCTAAGTTCTGAGCGGCCGCTTAATTAAGCTGCC | |
| | TTCTGCGGGGCTTGCCTTCTGGCCATGCCCTTCTTCTCTCCC | |
| | TTGCACCTGTACCTCTTGGTCTTTGAATAAAGCCTGAGTAGG | |
| | AAGTCTAG | |
| 9 | TAATACGACTCACTATA | T7 Promoter |

In some aspects, the polynucleotide construct of the disclosure is formulated with a delivery agent, e.g., a LNP.

### Delivery Agents

The delivery agents disclosed herein can effectively transport the polynucleotide constructs, cassettes, and mRNA disclosed herein into cells in vitro and in vivo.

In certain aspects, the delivery agent is a lipid nanoparticle, a liposome, a polymer, a micelle, a plasmids, a viral deliver agent, or any combination thereof.

Without being bound to any particular theory, the transport of polynucleotides constructs, expression cassettes, and/or mRNA disclosed herein by a delivery agents can occur via delivery of the polynucleotide construct to the cytosol of a cell. As gene expression and mRNA translation occurs in the cytosol of a cell, the polynucleotides have to enter the cytosol for effective modulation of the target gene or effective translation of a transported mRNA. If the polynucleotides do not enter the cytosol, they are likely to either be degraded or remain in the extracellular medium.

Examples of methods for the intracellular delivery of a biologically active polynucleotide to a target cell include those where the cell is in a mammalian animal, including, for example, a human, rodent, murine, bovine, canine, feline, sheep, equine, and simian mammal. In some aspects, the target cells for intracellular delivery are liver cells.

In some aspects, the delivery agent is a lipid nanoparticle (LNP). The polynucleotide constructs of the disclosure can be formulated within a LNP. In certain aspects, the polynucleotide construct is encapsulated within the LNP. “Encapsulated” as used herein refers containing a molecule, e.g., a polynucleotide, within the interior space of the LNP. In some aspects, by encapsulating the polynucleotide construct (e.g., comprising mRNA) within a delivery agent, such as a LNP, the nucleic acid (e.g., the polynucleotide construct of the disclosure) can be protected from an environment, which can contain enzymes or chemicals that degrade nucleic acids and/or systems or receptors that cause the rapid excretion of the nucleic acids. Lipid nanoparticles typically comprise an ionizable (e.g., cationic) lipid, a non-cationic lipid (e.g., cholesterol and a phospholipid), and a PEG lipid (e.g., a conjugated PEG lipid), which can be formulated with a payload of interest, e.g., a polynucleotide construct disclosed herein. The polynucleotide construct, e.g., mRNA, of the disclosure can be encapsulated in the lipid particle, thereby protecting it from enzymatic degradation. In some aspects, the molecule (e.g., a polynucleotide construct) is fully encapsulated by the LNP. In some aspects, at least 95%, at least 96%, at least 97%, at least 98%, at least 99%, or more of the molecule (e.g., a polynucleotide construct) in a lipid formulation is encapsulated by the LNP.

Certain aspects are directed to a composition comprising: a polynucleotide construct of the disclosure; and a delivery agent. The delivery agent can comprise an LNP, e.g., LNP compositions in LNP1 (PEG2000-C-DMA:13-B43:Cholesterol:DSPC), LNP2 (PEG2000-S:13-B43:Cholesterol:DSPC or PEG2000-S:18-B6:Cholesterol:DSPC), or LNP3 (PEG750-C-DLA:18-B6:Cholesterol:DSPC) groups.

In some aspects, the LNP of the disclosure comprises a PEG lipid selected from the group consisting of PEG2000-C-DMA, PEG2000-S, and PEG750-C-DLA. In some aspects, the LNP comprises a PEG lipid which is PEG2000-C-DMA. In some aspects, the LNP comprises a PEG lipid which is PEG2000-S. In some aspects, the LNP comprises a PEG lipid which is PEG750-C-DLA.

In some aspects, the LNP of the disclosure comprises an ionizable lipid which is 13-B43 or 18-B6.

In some aspects, the ionizable lipid is a compound of formula 13-B43, or a salt thereof. Such lipids are described, e.g., in WO 2013/126803 (PCT/US2013/027469).

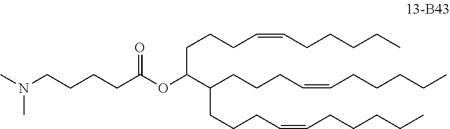 [SMILES: CCCCC/C=C\CCCC(CCC/C=C\CCCCC)C(CCC/C=C\CCCCC)OC(=O)CCCCN(C)C]

In some aspects, the ionizable lipid is a compound of formula 18-B6, or a salt thereof.

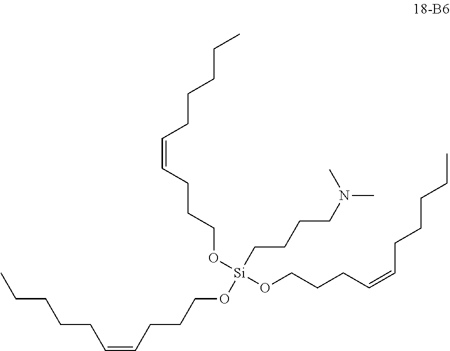 [SMILES: CCCCC/C=C\CCCO[Si](CCCCN(C)C)(OCCC/C=C\CCCCC)OCCC/C=C\CCCCC]

In some aspects, the LNP of the disclosure comprises a non-cationic lipid. In certain aspects the non-cationic lipid is a cholesterol, Distearoyl phosphatidylcholine (DSPC), or a combination thereof. In some aspects, the LNP comprises cholesterol. In some aspects, the LNP comprises Distearoyl phosphatidylcholine (DSPC). In some aspects, the LNP comprises cholesterol and Distearoyl phosphatidylcholine (DSPC).

In some aspects, the LNP of the disclosure comprises (a) a PEG Lipid (e.g., PEG2000-C-DMA, PEG2000-S, or PEG750-C-DLA); (b) an ionizable lipid (13-B43 or 18-B6); (c) a cholesterol; and (d) Distearoyl phosphatidylcholine (DSPC).

In certain aspects, the LNP of the disclosure comprises a PEG lipid in an amount of 0.1-4 mol %; 0.5-4 mol %, 2-3.5 mol %, 0.1-2 mol %; 0.5-2 mol %, or 1-2 mol % of the LNP. In certain aspects, the LNP comprises an ionizable lipid in an amount of 50-85 mol %; 50-65 mol %, or 50-60 mol % of the LNP. In certain aspects, the LNP comprises a non-cationic lipid in an amount of 45-50 mol % or up to about 50 mol %. In certain aspects, the LNP comprises a cholesterol in an amount of 30-40 mol % or 30-35 mol % of the LNP. In certain aspects, the LNP comprises an DSPC in an amount of 3-15 mol % or 6-12 mol % of the LNP.

In some aspects, the LNP of the disclosure comprises (a) 1-4 mol % PEG Lipid (e.g, PEG2000-C-DMA, PEG2000-S, or PEG750-C-DLA); (b) 50-60 mol % ionizable lipid (13-B43 or 18-B6); and (c) 45-50 mol % non-cationic lipid.

In some aspects, the LNP of the disclosure comprises (a) 1-4 mol % PEG Lipid (e.g, PEG2000-C-DMA, PEG2000-S, or PEG750-C-DLA); (b) 50-60 mol % ionizable lipid (13-B43 or 18-B6); (c) 30-35 mol % cholesterol; and (d) 6-12 mol % Distearoyl phosphatidylcholine (DSPC).

In some aspects, the size for LNPs are between about 50-200 nm in diameter. In some aspects, the LNP particle size ranges from about 50-150 nm, about 50-100 nm, about 50-120 nm, or about 50-90 nm.

### LNP Preparation

Those of skill in the art will appreciate that the following description is for illustration purposes only. The processes of the present disclosure are applicable to a wide range of lipid nanoparticle types and sizes. Further particles include, micelles, lipid-nucleic acid particles, virosomes, and the like. Those of skill in the art will know of other lipid LNPs for which the processes and apparatus of the present disclosure will be suitable.

In one aspect, the present method of encapsulating a polynucleic acid construct of the disclosure provides a lipid solution such as a clinical grade lipid synthesized under Good Manufacturing Practice (GMP), which is thereafter solubilized in an organic solution (e.g., ethanol). Similarly, a therapeutic product, e.g., a therapeutic active agent such as nucleic acid or other agent, is prepared under GMP. Thereafter, a therapeutic agent solution (e.g., mRNA) containing a buffer (e.g., citrate or ethanol) is mixed with a lipid solution solubilized in a lower alkanol to form a liposomal formulation. In preferred aspects of the disclosure, the therapeutic agent is “passively entrapped” in the liposome substantially coincident with formation of the liposome. However, those of skill in the art will realize that the processes and apparatus of the present disclosure are equally applicable to active entrapment or loading of the liposomes after formation of the LNP.

According to the processes and apparatus of the present disclosure, the action of continuously introducing lipid and buffer solutions into a mixing environment, such as in a mixing chamber, causes a continuous dilution of the lipid solution with the buffer solution, thereby producing a liposome substantially instantaneously upon mixing. As used herein, the phrase “continuously diluting a lipid solution with a buffer solution” (and variations) generally means that the lipid solution is diluted sufficiently rapidly in a hydration process with sufficient force to effectuate LNP generation. By mixing the aqueous solution with the organic lipid solution, the organic lipid solution undergoes a continuous stepwise dilution in the presence of the buffer (aqueous) solution to produce a liposome.

After the solutions, e.g., lipid solution and aqueous therapeutic agent (e.g., polynucleotide construct) solution, have been prepared, they are mixed together using, for example, a peristaltic pump mixer. In one aspect, the solutions are pumped at substantially equal flow rates into a mixing environment. In certain aspects, the mixing environment includes a “T”-connector or mixing chamber. In this instance, it is preferred that the fluid lines, and hence fluid flows, meet in a narrow aperture within the “T”-connector as opposing flows at approximately 180° relative to each other. Other relative introduction angles can be used, such as for example between 27° and 90° and between 90° and 180°. Upon meeting and mixing of the solution flows in the mixing environment, lipid LNPs are substantially instantaneously formed. Lipid LNPs are formed when an organic solution including dissolved lipid and an aqueous solution (e.g., buffer) are simultaneously and continuously mixed. Advantageously, and surprisingly, by mixing the aqueous solution with the organic lipid solution, the organic lipid solution undergoes a continuous stepwise dilution to substantially instantaneously produce a liposome. The pump mechanism can be configured to provide equivalent or different flow rates of the lipid and aqueous solutions into the mixing environment which creates lipid LNPs in a high alkanol environment.

Advantageously, the processes and apparatus for mixing of the lipid solution and the aqueous solution as provided herein provides for encapsulation of therapeutic agent in the formed liposome substantially coincident with liposome formation with an encapsulation efficiency of at least 90-95%. Further processing steps as discussed herein can be used to target a specific mRNA concentration by concentrating or diluting the sample, if desired.

In some aspects, the LNPs are formed having a mean diameter of less than about 150 nm (e.g., about 50-90 nm), which do not require further size reduction by high-energy processes such as membrane extrusion, sonication or microfluidization.

In certain aspects, LNPs form when lipids dissolved in an organic solvent (e.g., ethanol) are diluted in a stepwise manner by mixing with an aqueous solution (e.g., buffer). This controlled stepwise dilution is achieved by mixing the aqueous and lipid streams together in an aperture, such as a T-connector. The resultant lipid, solvent and solute concentrations can be kept constant throughout the LNP formation process.

In one aspect, using the processes of the present disclosure, a LNP is prepared by a two-stage step-wise dilution without gradients. For example, in the first stepwise dilution, LNPs are formed in a high alkanol (e.g., ethanol) environment (e.g., about 30% to about 50% v/v ethanol). These LNPs can then be stabilized by lowering the alkanol (e.g., ethanol) concentration to less than or equal to about 25% v/v, such as about 17% v/v to about 25% v/v, in a stepwise manner. In preferred aspects, with therapeutic agent present in the aqueous solution, or in the lipid solution, the therapeutic agent is encapsulated coincident with liposome formation.

In certain aspects, lipid stocks can be prepared in 100% ethanol, and then mixed with mRNA LNP in acetate buffer via a T-connector. The lipid and mRNA stocks can be mixed at a flow rate of 400 mL/min at the T-connector into a collection vessel containing PBS. In some aspects, lipids are initially dissolved in an alkanol environment of about 40% v/v to about 90% v/v, more preferably about 65% v/v to about 90% v/v, and most preferably about 80% v/v to about 90% v/v (A). Next, the lipid solution is diluted stepwise by mixing with an aqueous solution resulting in the formation of LNPs at an alkanol (e.g., ethanol) concentration of between about 37.5-50% (B). By mixing the aqueous solution with the organic lipid solution, the organic lipid solution undergoes a continuous stepwise dilution to produce a liposome. Further, lipid LNPs can be further stabilized by an additional stepwise dilution of the LNPs to an alkanol concentration of less than or equal to about 25%, preferably between about 15-25% (C).

In some aspects, for both stepwise dilutions (A→B and B→C), the resulting ethanol, lipid and solute concentrations are kept at constant levels in the receiving vessel. At these higher ethanol concentrations following the initial mixing step, the rearrangement of lipid monomers into bilayers proceeds in a more orderly fashion compared to LNPs that are formed by dilution at lower ethanol concentrations. Without being bound by any particular theory, it is believed that these higher ethanol concentrations promote the association of nucleic acid with cationic lipids in the bilayers. In certain aspects, the nucleic acid encapsulation occurs within a range of alkanol (e.g., ethanol) concentrations above 22%.

In certain aspects, after the lipid LNPs are formed, they are collected in another vessel, for example, a stainless steel vessel. In one aspect, a second dilution can be performed, e.g., at a rate of about 100-200 mL/min.

In one aspect, after the mixing step, the lipid concentration is about 1-10 mg/mL (e.g., about 7 mg/mL) and the therapeutic agent (e.g., mRNA) concentration is about 0.1-4 mg/mL.

After the mixing step, the degree of therapeutic agent (e.g., nucleic acid) encapsulation can be enhanced if the lipid LNP suspension is optionally diluted. For example, prior to dilution step, if the therapeutic agent entrapment is at about 30-40%, it can be increased to about 70-80% following incubation after the dilution step. In step, the liposome formulation is diluted to about 10% to about 40%, preferably about 20% alkanol, by mixing with an aqueous solution such as a buffer (e.g., PBS). Such further dilution is preferably accomplished with a buffer. In certain aspects, such further diluting the liposome solution is a continuous stepwise dilution. The diluted sample is then optionally allowed to incubate at room temperature.

After the optional dilution step, about 70-80% or more of the therapeutic agent (e.g., nucleic acid) is entrapped within the lipid LNP. In certain aspects, anion exchange chromatography is used.

In certain instances, the liposome solution is optionally concentrated about 2-6 fold, preferably about 4 fold, using for example, ultrafiltration (e.g., tangential flow dialysis). In one aspect, the sample is transferred to a feed reservoir of an ultrafiltration system and the buffer is removed. The buffer can be removed using various processes, such as by ultrafiltration.

In some aspects, the concentrated formulation is then diafiltrated to remove the alkanol. The alkanol concentration at the completion of step is less than about 1%. Preferably, lipid and therapeutic agent (e.g., nucleic acid) concentrations remain unchanged and the level of therapeutic agent entrapment also remains constant.

After the alkanol has been removed, the aqueous solution (e.g., buffer) is then replaced by dialfiltration against another buffer. Preferably, the ratio of concentrations of lipid to therapeutic agent (e.g., nucleic acid) remain unchanged and the level of nucleic acid entrapment is about constant. In certain instances, sample yield can be improved by rinsing the cartridge with buffer at about 10% volume of the concentrated sample. In certain aspects, this rinse is then added to the concentrated sample.

In certain aspects, sterile filtration of the sample can optionally be performed. In certain aspects, filtration is conducted at pressures below about 40 psi, using a capsule filter and a pressurized dispensing vessel with a heating jacket. Heating the sample slightly can improve the ease of filtration.

The sterile fill step can be performed using a processes for conventional liposomal formulations. In some aspects, the processes of the present disclosure results in about 50-60% of the input therapeutic agent (e.g., nucleic acid) in the final product. In certain preferred aspects, the therapeutic agent to lipid ratio of the final product is approximately 0.04 to 0.07.

Preparation of encapsulated LNPs can then be filtered under sterile conditions, aliquoted, and stored at −80° C.

### Copolymers

In some aspects, the composition of the disclosure further comprises a copolymer. In some aspects, the copolymer disclosed herein is a “membrane destabilizing polymers” or “membrane disruptive polymers.” Membrane destabilizing polymers or membrane disruptive polymers can directly or indirectly elicit a change, such as a permeability change for example, in a cellular membrane structure, such as an endosomal membrane for example, so as to permit an agent, for example an oligonucleotide or copolymer or both, to pass through such membrane structure. In some aspects, the membrane disruptive polymer can directly or indirectly elicit lysis of a cellular vesicle or otherwise disrupt a cellular membrane for example as observed for a substantial fraction of a population of cellular membranes.

The delivery agents, copolymers and compositions as disclosed herein can be useful in methods for the intracellular delivery of the polynucleotide constructs of the disclosure, to target cells, including target cells in vitro, ex vivo, and in vivo. In some aspects, a method of delivering a polynucleotide constructs, e.g., comprising an mRNA, to a target cell includes delivery to the cytosol of the cell.

### Compositions

The delivery agents disclosed herein can effectively transport polynucleotide constructs into cells both in vitro and in vivo. In some aspects, the polynucleotide construct of the disclosure is formulated with a delivery agent, e.g., an LNP. In some aspects, the compositions further comprises a pharmaceutically acceptable carrier.

Certain aspects of the disclosure are directed to a composition or method for increasing the amount of the OTC protein in a cell. In some aspects, the polynucleotide construct comprising a nucleic acid sequence comprising a codon optimized mRNA sequence comprising an open reading frame (ORF) encoding a functional human ornithine transcarbamylase (OTC) is formulated with an LNP and/or a copolymer into a composition. In certain aspects, the mRNA molecule encodes an OTC protein comprising an amino acid sequence having at least 85%, at least 90%, at least 95%, at least 96%, at least 97%, at least 98%, at least 99%, or 100% sequence identity with SEQ ID NO:7. To direct an encoded OTC protein to the mitochondria of the cell, the mRNA molecule encoding the OTC protein can include a sequence encoding a mitochondrial targeting signal peptide (also referred to herein as a “mitochondrial leader sequence”). The mitochondrial leader sequence can be that of a native OTC protein (e.g., comprising residues 1-32 of SEQ ID NO:7 (a native human mitochondrial leader sequence), or can be derived from another protein comprising a mitochondrial targeting signal peptide, or synthesized de novo. An engineered cleavage site can be included at the junction between the mitochondrial leader sequence and the remainder of the polypeptide to optimize proteolytic processing in the cell. The mitochondrial leader sequence is operably linked to the mRNA sequence encoding the mature OTC protein, i.e., the two sequences are joined in the correct reading frame and positioned to direct the newly synthesized polypeptide to the mitochondria of a cell. Mitochondrial leader sequences are commonly positioned at the amino terminus of the protein. In specific variations, the encoded OTC protein with a mitochondrial leader sequence has an amino acid sequence as set forth in SEQ ID NO: 7. Suitable mRNA sequences encoding an OTC protein of SEQ ID NO:7, and which can be formulated into a composition of the present disclosure, can comprise sequences as shown in SEQ ID NO:1 or SEQ ID NO:4. Suitable mRNA sequences encoding an OTC protein of SEQ ID NO:7, and which can be formulated into a composition of the present disclosure, can comprise a sequence as shown in SEQ ID NO: 1 or SEQ ID NO: 4. An OTC-encoding mRNA for formulation in the present disclosure typically further includes a poly(A) at its 3′ end (e.g., a polyA tail of greater than 80, e.g., 100 to 800 adenine residues), which can be added to a construct using well-known genetic engineering techniques (e.g., via PCR or enzymatic Poly-A tail). Exemplary DNA sequences that can be used for insertion into an appropriate DNA vector for production and preparation of the polynucleotide constructs of the disclosure.

### Methods of Use

Certain aspects of the disclosure are directed to increasing the amount of ornithine transcarbamylase (OTC) in a cell by contacting the cell with a composition comprising a polynucleotide construct disclosed herein and a pharmaceutically acceptable diluent or carrier. In some aspects, the polynucleotide construct is formulated with an LNP disclosed herein. In further aspects, the polynucleotide can be formulated with a copolymer.

Some aspects are directed to a method for increasing the amount of OTC expression in a cell comprising administering to the cell a composition comprising the polynucleotide construct of the disclosure. The cell can be a liver cell.

A method for treating ornithine transcarbamylase deficiency (OTCD) comprising administering to a subject in need thereof a therapeutically effective amount of a composition comprising the polynucleotide construct of the disclosure.

A method for treating or reducing the risk of hyperammonemia in a subject with OTCD comprising administering to a subject in need thereof a therapeutically effective amount of a composition comprising the polynucleotide construct of the disclosure.

Other aspects of the disclosure are directed to the use of a polynucleotide constructs of the disclosure or composition of the disclosure, or a vector of the disclosure, or a host cell of the disclosure, for the manufacture of a medicament for the treatment of OTCD in a subject in need thereof or for the manufacture of a medicament for the treatment or reducing the risk of hyperammonemia in a subject with OTCD.

A disease or condition associated with defective gene expression and/or activity in a subject treatable by the methods disclosed herein includes ornithine transcarbamylase deficiency (OTCD).

In certain aspects, the disease or condition associated with defective gene expression is a disease characterized by a deficiency in a functional polypeptide (also referred to herein as a “disease associated with a protein deficiency”). A delivery agent, e.g., LNP, of the disclosure can be formulated into a composition comprising a messenger RNA (mRNA) molecule encoding a protein corresponding to a genetic defect that results in a deficiency of the protein. For treatment of the disease associated with the protein deficiency, the polynucleic acid construct, e.g., comprising an mRNA, formulation can be administered to a subject (e.g., mammal such as, for example, a mouse, non-human primate, or human) for delivery of the mRNA to an appropriate target tissue, where the mRNA is translated during protein synthesis and the encoded protein is produced in an amount sufficient to treat the disease.

An example of a method of treating a disease or condition associated with defective gene expression and/or activity in a subject, such as a mammal for example, includes administering to a mammal in need thereof a therapeutically effective amount of a polynucleotide construct comprising a nucleic acid sequence comprising a codon optimized mRNA sequence comprising an open reading frame (ORF) encoding a functional human ornithine transcarbamylase (OTC) is formulated with an LNP and/or a copolymer into a composition. In certain aspects, the mRNA molecule encodes an OTC protein comprising an amino acid sequence having at least 85%, at least 90%, at least 95%, at least 96%, at least 97%, at least 98%, at least 99%, or 100% sequence identity with SEQ ID NO:7. To direct an encoded OTC protein to the mitochondria of the cell, the mRNA molecule encoding the OTC protein can include a sequence encoding a mitochondrial targeting signal peptide (also referred to herein as a “mitochondrial leader sequence”). The mitochondrial leader sequence can be that of a native OTC protein (e.g., comprising residues 1-32 of SEQ ID NO:7 (a native human mitochondrial leader sequence), or can be derived from another protein comprising a mitochondrial targeting signal peptide, or synthesized de novo. An engineered cleavage site can be included at the junction between the mitochondrial leader sequence and the remainder of the polypeptide to optimize proteolytic processing in the cell. The mitochondrial leader sequence is operably linked to the mRNA sequence encoding the mature OTC protein, i.e., the two sequences are joined in the correct reading frame and positioned to direct the newly synthesized polypeptide to the mitochondria of a cell. Mitochondrial leader sequences are commonly positioned at the amino terminus of the protein. In specific variations, the encoded OTC protein with a mitochondrial leader sequence has an amino acid sequence as set forth in SEQ ID NO: 7. Suitable mRNA sequences encoding an OTC protein of SEQ ID NO:7, and which can be formulated into a composition of the present disclosure, can comprise sequences as shown in SEQ ID NO:1 or SEQ ID NO:4. Suitable mRNA sequences encoding an OTC protein of SEQ ID NO:7, and which can be formulated into a composition of the present disclosure, can comprise a sequence as shown in SEQ ID NO: 1 or SEQ ID NO: 4. An OTC-encoding mRNA for formulation in the present disclosure typically further includes a poly(A) at its 3′ end (e.g., a polyA tail of greater than 80, e.g., 100 to 800 adenine residues).

A further example of a method for treating a disease or condition associated with defective gene expression includes a method of treating a subject having a deficiency in a functional polypeptide comprising administering to the subject a composition comprising at least one mRNA molecule at least a portion of which encodes the functional polypeptide where following administration the expression of the functional polypeptide is greater than before administration. In some aspects, the mRNA encodes a functional ornithine transcarbamylase (OTC) protein.

In particular variations, a composition comprising an mRNA encoding an ornithine transcarbamylase (OTC) protein is used in a method to treat ornithine transcarbamylase deficiency (OTCD). OTCD is a urea cycle disorder that can trigger hyperammonemia, a life-threatening illness that leads to brain damage, coma or even death. This is due to deficiency in the activity of OTC, a key enzyme in the urea cycle, which primarily takes place in the liver and is responsible for removal of ammonia from the body. Ammonia is produced from protein intake as well as protein breakdown in the body. In the liver, this ammonia is converted into urea by enzymes in the urea cycle. Urea is non-toxic and cleared easily through the kidneys in urine, normally. However, when the OTC enzyme is deficient, ammonia levels rise in blood and can cause severe brain damage. Patients with severe OTC deficiency are most often identified 2-3 days after birth where the patient has significantly elevated blood ammonia levels and ends up in a coma. Patients with milder OTC deficiency can have crises during times of stress resulting in elevated ammonia levels that can also lead to coma. Current therapies include ammonia scavenger drugs (Buphenyl, Ravicti) for use in patients with hyperammonemia.

The OTC gene is X-linked. The disease is present in males with one mutant allele and in females either homozygous or heterozygous with mutant alleles. Male patients with the severest OTC deficiency are typically found right after birth. In addition to elevation in blood ammonia levels, urinary orotic acid levels are also elevated. In patients with severe OTC deficiency, OTC enzyme activity is <2% of normal levels. In patients with milder OTC deficiency, OTC enzyme activity is up to 30% of normal levels.

A method for treating OTCD with a polynucleotide construct of the disclosure or composition comprising an OTC-encoding mRNA of the present disclosure generally includes administering to a subject having OTCD a therapeutically effective amount of the composition, whereby the OTC-encoding mRNA is delivered to liver cells and translated during protein synthesis to produce the OTC protein. The OTC-encoding mRNA can be an mRNA as set forth above with respect to a composition or method for increasing OTC protein in a cell.

The efficacy of an mRNA composition for treating a disease can be evaluated in vivo in animal models of disease. For example, suitable animal models for evaluating efficacy of an mRNA composition for treatment of OTCD includes known mouse models having deficiencies of the OTC enzyme in the liver. One such mouse model, Otc^{spf-ash} (sparse fur and abnormal skin and hair) mice, contain an R129H mutation resulting in reduced levels of OTC protein and have only 5-10% of the normal level of enzyme activity in liver (see Hodges et al., PNAS 86:4142-4146, 1989). Another model, Otc^{spf} mice, contain an H117N mutation which results in reduced levels of enzyme activity to 5-10% of normal levels (see Rosenberg et al., Science 222:426-428, 1983). Both of these mouse models have elevated urine orotic acid levels compared to their wild-type littermate mice. A third model for OTC deficiency is inducing hyperammonemia in Otc^{spf} or Otc^{spf-ash} mice (Cunningham et al., Mol Ther 19(5): 854-859, 2011). These mice are treated with OTC siRNA or AAV2/8 vector/OTC shRNA to knockdown residual endogenous OTC expression and activity. Plasma ammonia levels are elevated and mice die approximately 2-14 days.

Once the detection of specific analytes narrows the diagnostic possibilities, the activity of the deficient enzyme is assayed in lymphocytes or cultured fibroblasts as a confirmatory test. For many pathways, no single enzyme assay can establish the diagnosis. For others, tests such as complementation studies need to be done.

In certain aspects, the goal of therapy is to restore biochemical and physiologic homeostasis. Neonates may require emergency diagnosis and treatment depending on the specific biochemical lesion, the position of the metabolic block, and the effects of the toxic compounds. Treatment strategies include: (1) dietary restriction of the precursor amino acids and (2) use of adjunctive compounds to (a) dispose of toxic metabolites or (b) increase activity of deficient enzymes. Liver transplantation has been successful in a small number of affected individuals. Even with current clinical management approaches, individuals with organic acidemias have a greater risk of infection and a higher incidence of pancreatitis, which can be fatal.

In certain aspects, the polynucleotide constructs and compositions of the present disclosure is useful in the preparation of a medicament for the treatment of a disease or condition associated with defective gene expression and/or activity in a subject.

The polynucleotide constructs and compositions of the present disclosure can be administered in a variety of routes of administration such as parenteral, oral, topical, rectal, inhalation and the like. Formulations will vary according to the route of administration selected. In some aspects, the route of administration is intravenously, intramuscularly, intracutaneously, subcutaneously, intraduodenally, or intraperitoneally.

Determination of the proper dosage for a particular situation is within the skill of the art. Effective doses of the compositions of the present disclosure vary depending upon many different factors, including means of administration, target site, physiological state of the patient, whether the patient is human or an animal, other medications administered, as well as the specific activity of the composition itself and its ability to elicit the desired response in the individual. Usually, the patient is a human, but in some diseases, the patient can be a nonhuman mammal.

### EXAMPLES

### Example 1. Preparation of OTC Polynucleotide Constructs

An OTC polynucleotide constructs comprising the sequence of SEQ ID NO: 4 were prepared by In Vitro Transcription (IVT) using a plasmid DNA construct. The plasmid DNA construct contained the instructions for the 5′UTR, ORF and 3′UTR while the chemical modification (e.g. Pseudouridine) was determined by the addition of the desired nucleotide to the IVT reaction. To start, the plasmid DNA was linearized using 5 units of XbaI restriction enzyme per ug of plasmid DNA. After an overnight incubation at 37 degrees the DNA was purified by phenol/chloroform extraction. An IVT reaction in addition to co-trascriptional capping (e.g., Cap1) was performed for 3 hours at 37 degrees using T7 Polymerase and CleanCap. After the IVT reaction, the resultant mRNA product was purified via DNase treatment followed by Diafiltration. The purified mRNA was then enzymatically Poly adenylated with 300 units of Poly A polymerase per mg RNA and incubated for between 15 and 60 minutes, depending on the desired Poly A tail length. The mRNA product was then purified by Diafiltration and HPLC before being adjusted to a desired concentration, sterile filtered and aliquoted.

### Example 2. Effects of Poly(A) Tail Length on Potency and Tolerability

OTC mRNA constructs as described in Example 1 were prepared with a poly(A) tails having variable lengths. In a first experiment, OTC mRNA was transcribed and the crude transcript was used as a template for a reaction with pre-warmed or cold PolyA polymerase. In a second experiment, OTC mRNA was transcribed, purified, and the purified transcript was used as a template for a reaction with pre-warmed or cold PolyA polymerase. In a third experiment, the reaction time to yield the correct PolyA tail length was determined.

PolyA experiments 1 and 2 resulted in no significant difference in the length of PolyA tails generated. Additionally, enzyme temperature did not affect run performance. In experiments 1 and 2, the reaction time was 30 min. In experiment 3, reaction times of 45, 60, and 75 min were tested. 60 and 75 minute reaction times were able to generate PolyA tails over 300 nucleotides (nts) in length. Although the longer reaction times produced longer tails, the reaction time also impacted the purity of the product.

To assess the effect of different poly(A) tail length (encoded or enzymatic) on potency and tolerability, a rat repeated dose study was performed. An OTC construct comprising mRNA with different poly(A) tail lengths (80, 161, 208, 262, 322, or 440 nts) encapsulated in LNP2 (PEG2000-S:13-B43:Cholesterol:DSPC) was administered to male Srague Dawley rats (7-8 weeks old) at DO, 7, and 14 (Table 2A). The experiment was terminated at D1 (24 h post-dose) or D15 (24 h post-last-dose). The Z-Avg, PDI, and % Encaps of each formulation administered is provided in Table 2B. All formulations were tested for endotoxin by in-house LAL assay. All formulations were below 2 EU/mL when at 0.5 mg/mL.

TABLE 2A
Administration and Dosing of LNP2 Formulations

| | | | | Dose | | |
|---|---|---|---|---|---|---|
| | | | | (mg/ | Dose | |
| Group | N | LNP | Payload | kg) | Day | Terminal |
| 1a/ | 4 | PBS | — | — | D0 | N = 2 on D1 (24 h) |
| 1b | | | | | (n = 2) | N = 2 on D15 (24 h) |
| | | | | | D0, | |
| | | | | | 7, 14 | |
| | | | | | (n = 2) | |
| 2 | 4 | LNP2 | OTC mRNA | 0.25 | D0 | D1 (24 h post-dose) |
| 3 | 4 | (13- | Construct-80 | | D0, | D15 (post-last dose) |
| | | (B43) | nts Poly(A) | | 7, 14 | |
| | | | encoded | | | |
| 4 | 4 | | OTC mRNA | | D0 | D1 (24 h post-dose) |
| 5 | 4 | | Construct-161 | | D0, | D15 (post-last dose) |
| | | | nts Poly(A) | | 7, 14 | |
| 6 | 4 | | OTC mRNA | | D0 | D1 (24 h post-dose) |
| 7 | 4 | | Construct-208 | | D0, | D15 (post-last dose) |
| | | | nts Poly(A) | | 7, 14 | |
| 8 | 4 | | OTC mRNA | | D0 | D1 (24 h post-dose) |
| 9 | 4 | | Construct-262 | | D0, | D15 (post-last dose) |
| | | | nts Poly(A) | | 7, 14 | |
| 10 | 4 | | OTC mRNA | | D0 | D1 (24 h post-dose) |
| 11 | 4 | | Construct-322 | | D0, | D15 (post-last dose) |
| | | | nts Poly(A) | | 7, 14 | |
| 12 | 4 | | OTC mRNA | | D0 | D1 (24 h post-dose) |
| 13 | 4 | | Construct-440 | | | |
| | | | nts Poly(A) | | D0, | D15 (post-last dose) |
| | | | | | 7, 14 | |

TABLE 2B
LNP2 Formulation Characteristics

| Formulation | Poly(A) Length | Z-Avg (nm) | PDI | % Encapsulation |
|---|---|---|---|---|
| LNP2 (13-B43) | 80 | 86 | 0.08 | 96 |
| | 208 | 76 | 0.08 | 97 |
| | 161 | 76 | 0.06 | 97 |
| | 262 | 79 | 0.06 | 95 |
| | 322 | 83 | 0.04 | 97 |
| | 440 | 79 | 0.05 | 96 |

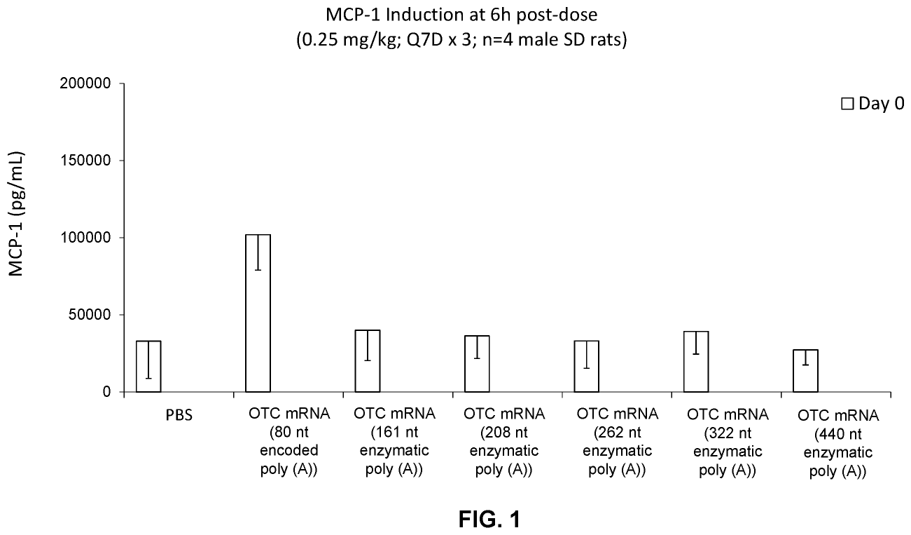

Monocyte Chemoattractant Protein-1 (MCP-1) induction levels 6 h after the first dose were analyzed for various polyA constructs, and the results are shown in FIG. 1.

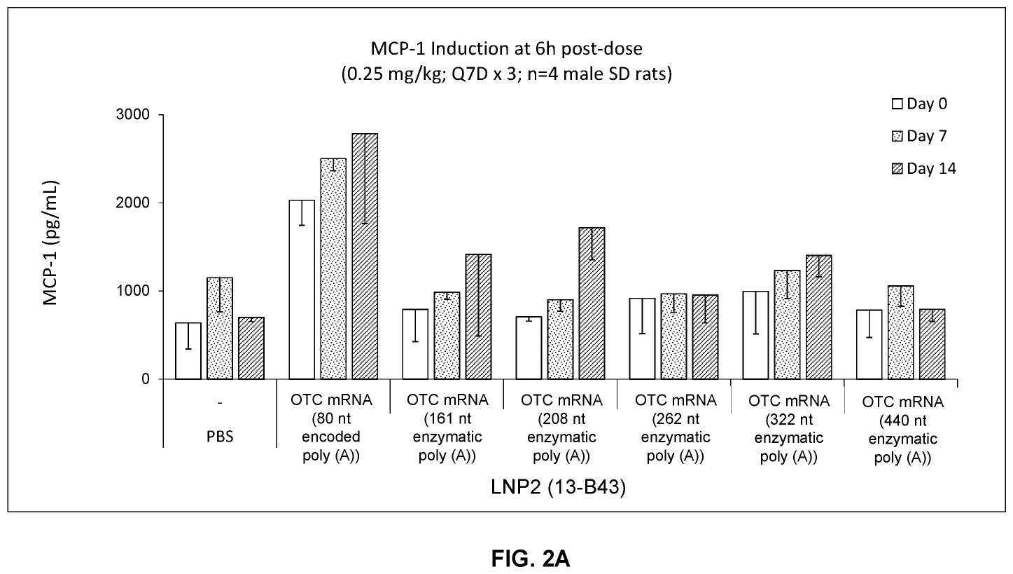

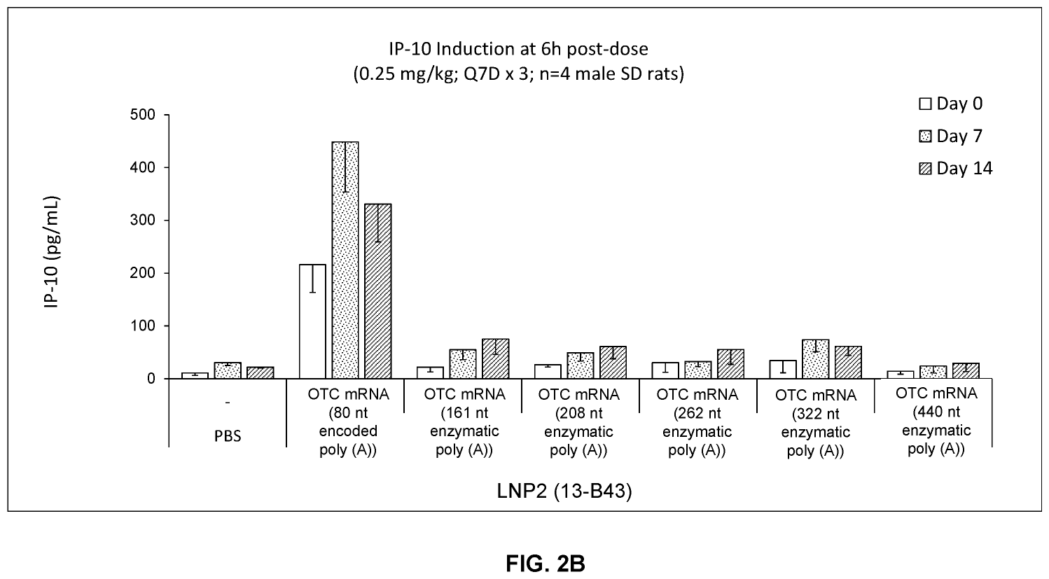

To analyze the induction of immune responses to administration of the LNPs formulated with OTC constructs including mRNA with various polyA tail lengths upon repeat dosing, tail pokes were obtained 6 h after dosing on each dosing day and rat cytokine induction was quantified. Monocyte Chemoattractant Protein-1 (MCP-1) induction levels 6 h after dosing (Day 0, Day 7, and 14) were analyzed (FIG. 2A). The OTC mRNA having a 80 nts encoded Poly(A) resulted in higher MCP-1 induction levels compared to OTC mRNA constructs having 161, 208, 262, 322, or 440 nt enzymatic Poly(A) tails. MCP-1 and interferon γ-induced protein 10 (IP-10) induction levels were analyzed at 6 h post-dosing on days DO, D7, and D14 (FIG. 2B). All responses were compared to PBS control group. The OTC mRNA construct with 80 nt encoded Poly(A) tail showed higher MCP-1 (FIG. 2A) and IP-10 (FIG. 2B) induction compared to the tested OTC mRNA constructs with enzymatic Poly(A) tails greater than 80 nucleotides.

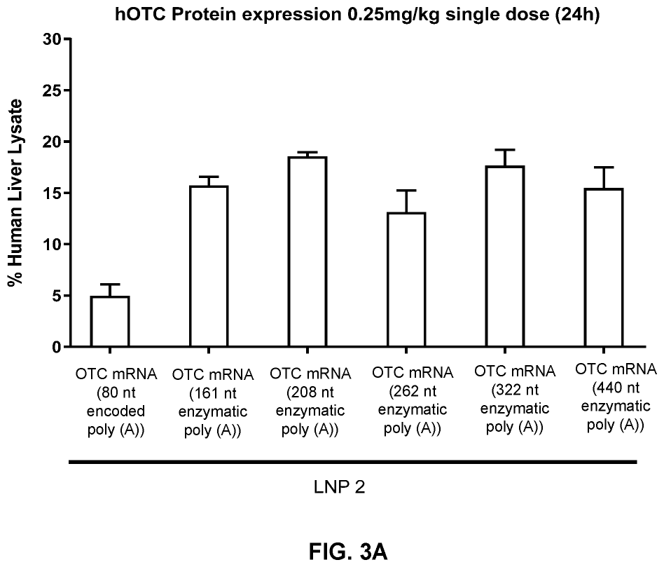

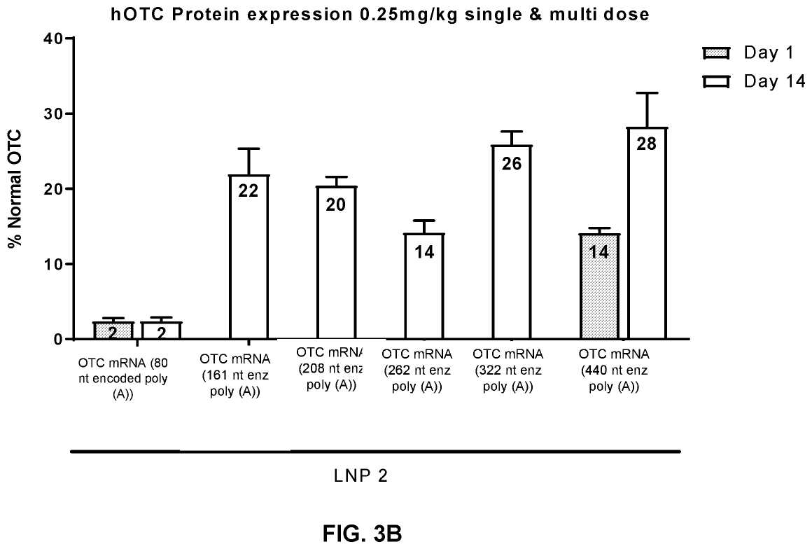

To analyze OTC protein expression, rat liver samples were obtained 24 hr post-last-dose and flash frozen. The OTC construct having the 80 nucleotide encoded Poly(A) had the lowest hOTC protein expression in the liver compared the OTC constructs having the enzymatic Poly(A) tails greater than 80 nucleotides (FIG. 3A and FIG. 3B).

### Example 3. Modified OTC mRNA Constructs

To assess the effect of chemical modifications on potency and tolerability, a mouse study was performed. OTC mRNA prepared in Example 1 (having a polyA tail range ˜180-480 nucleotides long) was chemically modified with either pseudouridine (PsU), N1-methyl-pseudouridine (N1MePsU), or 5-methoxyduridine (5MoU) (Table 3A) using TriLink methods.

The chemically modified mRNA was formulated into either LNP1 or LNP2 (PEG2000-S:13-B43:Cholesterol:DSPC) (Table 3B) and administered to mice (0.5 mg/kg) (Table 3C).

TABLE 3A
Chemical Modification of mRNAs

| Group | N | Composition | mRNA & Mods | Dose | |
|---|---|---|---|---|---|
| 1 | 4 | PBS | | 10 | mL/kg |
| 2 | 4 | LNP1 | OTC mRNA-PsU | 0.5 | mg/kg |

| | | | |
|---|---|---|---|
| 3 | 4 | LNP2 (13-B43) | |
| 4 | 4 | LNP1 | OTC mRNA- |
| 5 | 4 | LNP2 (13-B43) | N1MePsU |
| 6 | 4 | LNP1 | OTC mRNA-5MoU |
| 7 | 4 | LNP2 (13-B43) | |

TABLE 3B
LNP Formulation of Chemically Modified mRNAs

| | | | mRNA | | | |
|---|---|---|---|---|---|---|
| | | | length | Poly(A) | Expected | |
| | | | before | tail | Total | ~Actual |
| | | Purity | PAP | length | length | length |
| Variant | Purification | (%) | (nt) | (nt) | (nt) | (CE) |
| PsU | HPLC | 93 | 1226 | 322 | 1548 | 1600 |
| 5MoU | Silica | 85 | 1226 | 186 | 1412 | 1700 |
| N1MePsU | HPLC | 90 | 1226 | 470 | 1696 | 1800 |

TABLE 3C
Administration of Chemically Modified mRNA

| | | Z-Avg | | Encaps | Conc |
|---|---|---|---|---|---|
| LNP | Payload | (nm) | PDI | (%) | (mg/mL) |
| LNP1 | OTC mRNA- | 78 | 0.09 | 96 | 0.045 |
| LNP2 | PsU | 84 | 0.12 | 96 | 0.045 |
| (13-B43) | | | | | |
| LNP1 | OTC mRNA- | 73 | 0.06 | 96 | 0.048 |
| LNP2 | N1MePsU | 77 | 0.09 | 96 | 0.048 |
| (13-B43) | | | | | |
| LNP1 | OTC mRNA- | 73 | 0.07 | 96 | 0.048 |
| LNP2 | 5MoU | 77 | 0.09 | 95 | 0.050 |
| (13-B43) | | | | | |

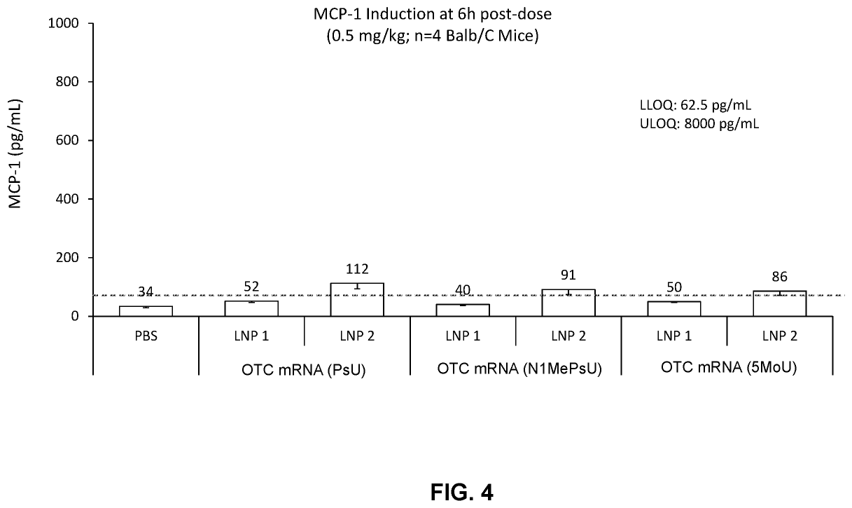

MCP-1 levels were analyzed after administration of the modified OTC mRNA formulations (FIG. 4). There were no significant differences in MCP-1 response between the different tested OTC mRNA chemical modifications. LNP2 (PEG2000-S:13-B43:Cholesterol:DSPC) was slightly more stimulatory compared to LNP1.

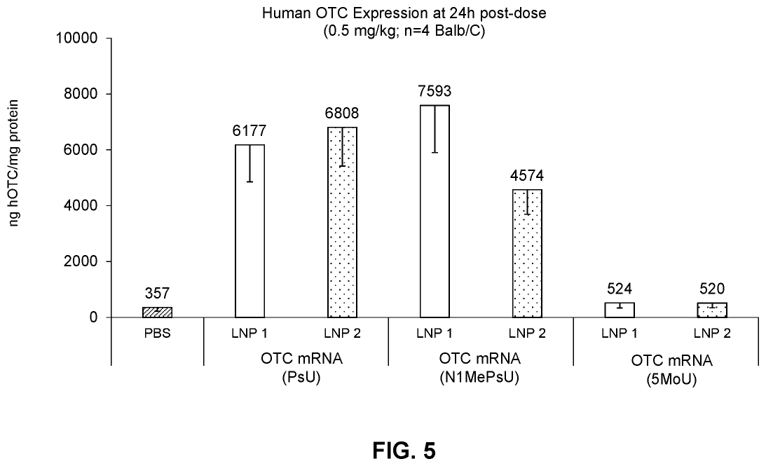

Next, human OTC expression was analyzed by ELISA (FIG. 5). There were similar levels of OTC expression between OTC mRNA PsU and N1MePsU modifications in both LNPs. The lowest OTC expression was detected in OTC mRNA 5MoU-LNP treated animals. OTC mRNA N1MePsU-LNP1 treated animals had higher OTC expression than OTC mRNA PsU-LNP1 treated animals. OTC mRNA PsU-LNP2 treated animals had higher OTC expression than OTC mRNA N1MePsU treated animals.

### Example 4. OTC mRNA-LNP Tolerability and OTC Expression in Rats

OTC mRNA-PsU potency and tolerability was evaluated in a rat repeat dose study. OTC mRNA-PsU (0.25 mg/kg) was formulated in either LNP1 (PEG2000-C-DMA: 13-B43:Cholesterol:DSPC), LNP2 (PEG2000-S:13-B43:Cholesterol:DSPC or PEG2000-S:18-B6:Cholesterol:DSPC), or LNP3 (PEG750-C-DLA:18-B6:Cholesterol:DSPC) and administered to mice on Day 0, 7, and 14 (Table 4A). EPO and LUC were carried in LNP1 and administered as controls.

TABLE 4A
Administration and Dosing of OTC mRNA Construct-PsU

| | | | | Dose | | |
|---|---|---|---|---|---|---|
| Group | N | LNP | Payload | (mg/kg) | Dose Day | Terminal |
| 1 | 2 | PBS | — | — | D0 | D1 (24 h) |
| 2 | 3 | LNP1 | EPO | 0.25 | D0, 7, 14 | D14 (24 h) |
| | | | (5MoU) | | | |
| 3 | 3 | | LUC | | D0, 7, 14 | D14 (24 h) |
| | | | (5MoU) | | | |
| 4 | 3 | | OTC | | D0 | D1 (24 h |
| | | | mRNA | | | post-last |
| | | | (262 nts | | | dose) |
| | | | Poly(A)) | | | |
| | | | (PsU) | | | |
| 5 | 3 | | OTC | | D0, 7, 14 | D15 (24 h |
| | | | mRNA | | | post-last |
| | | | (262 nts | | | dose) |
| | | | Poly(A)) | | | |
| | | | (PsU) | | | |
| 6 | 3 | LNP2 (13- | OTC | | D0 | D1 (24 h |
| | | B43) | mRNA(262 | | | post-last |
| | | | nts | | | dose) |
| 7 | 3 | | Poly(A)) | | D0, 7, 14 | D15 (24 h |
| | | | (PsU) | | | post-last |
| | | | | | | dose) |
| 8 | 3 | LNP2 (18- | | | D0 | D1 (24 h |
| | | B6) | | | | post-last |
| | | | | | | dose) |
| 9 | 3 | | | | D0, 7, 14 | D15 (24 h |
| | | | | | | post-last |
| | | | | | | dose) |
| 10 | 3 | LNP3 | | | D0 | D1 (24 h |
| | | | | | | post-last |
| | | | | | | dose) |
| 11 | 3 | | | | D0, 7, 14 | D15 (24 h |
| | | | | | | post-last |
| | | | | | | dose) |

The Z-Avg, PDI, and % Encaps of each formulation administered is provided in Table 4B. Input batch size was 3 mg. LNPs were formulated with 100 mM acetate, pH5 and worked up on TFU. Aliquots were stored at −80° C. and test articles were prepared on each day of dosing.

TABLE 4B
LNP1, LNP2, and LNP3 Formulation Characteristics

| LNP | | Z-avg | | Encaps | Recovery | |
|---|---|---|---|---|---|---|
| Composition | Payload | (nm) | PDI | (%) | (%) | Endotoxin |
| LNP1 | Human | 74 | 0.15 | 98 | 84 | <5 EU/mL |
| | EPO | | | | | |
| LNP1 | LUC | 71 | 0.10 | 96 | 74 | <5 EU/mL |
| LNP1 | OTC | 74 | 0.09 | 96 | 74 | <5 EU/mL |
| | mRNA | | | | | |
| | (262 nts | | | | | |
| | Poly(A)) | | | | | |
| LNP2 (13- | OTC | 74 | 0.11 | 96 | 76 | <5 EU/mL |
| B43) | mRNA | | | | | |
| | (262 nts | | | | | |
| | Poly(A)) | | | | | |
| LNP2 (18- | OTC | 71 | 0.10 | 94 | 78 | <5 EU/mL |
| B6) | mRNA | | | | | |
| | (262 nts | | | | | |
| | Poly(A)) | | | | | |
| LNP3 | OTC | 76 | 0.13 | 90 | 74 | <5 EU/mL |
| | mRNA | | | | | |
| | (262 nts | | | | | |
| | Poly(A)) | | | | | |

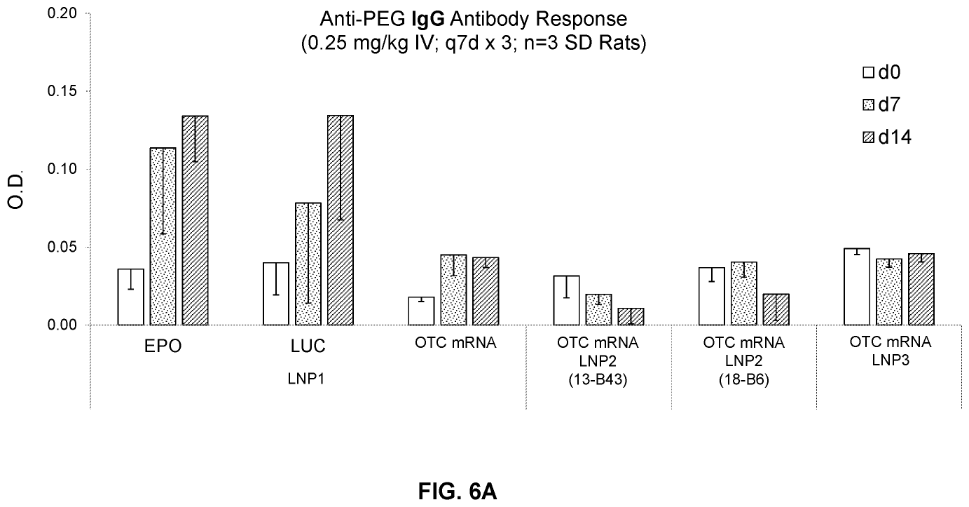

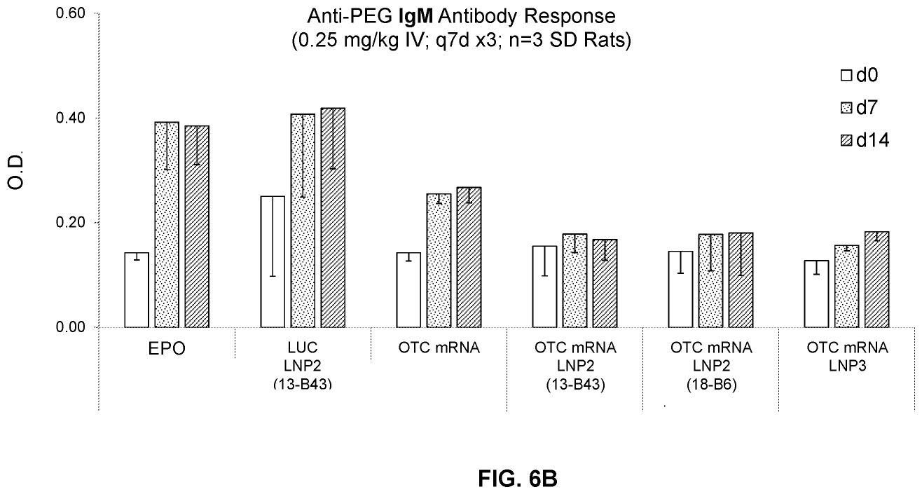

To examine PEG-antibody levels, blood was collected pre-dose on each dosing day (DO, 7, and 14). Both anti-PEG IgG (FIG. 6A) and anti-PEG IgM (FIG. 6B) antibody responses were quantified. Anti-PEG antibodies were observed in rats treated with LNP1 only. The tested OTC mRNA constructs were less immunogenic than the EPO and LUC payloads. Generation of anti-PEG antibodies with LNP1 resulted in accelerated blood clearance and loss of potency upon repeated dose (data not shown).

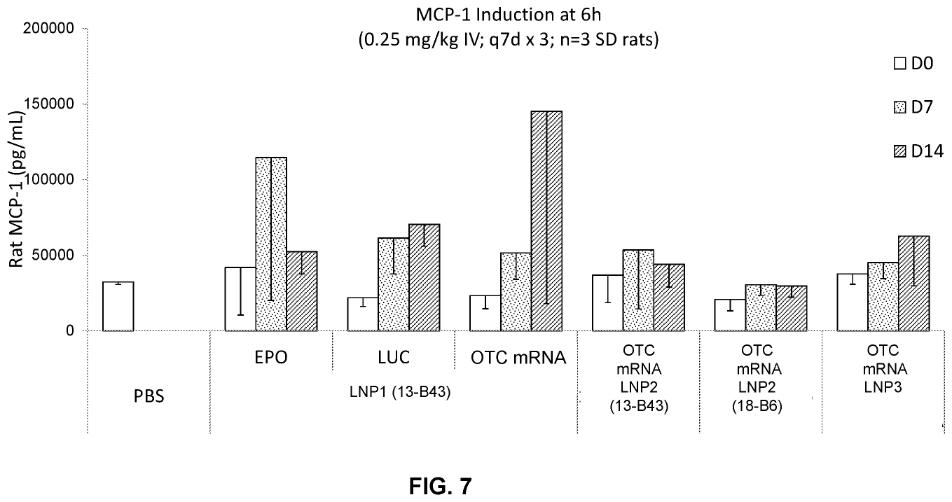

To examine MCP-1 induction, blood was collected 6 h after each dosing. There was little to no increase in MCP-1 upon repeat dose of LNP containing OTC mRNA constructs which correlates with lower immunogenicity (FIG. 7).

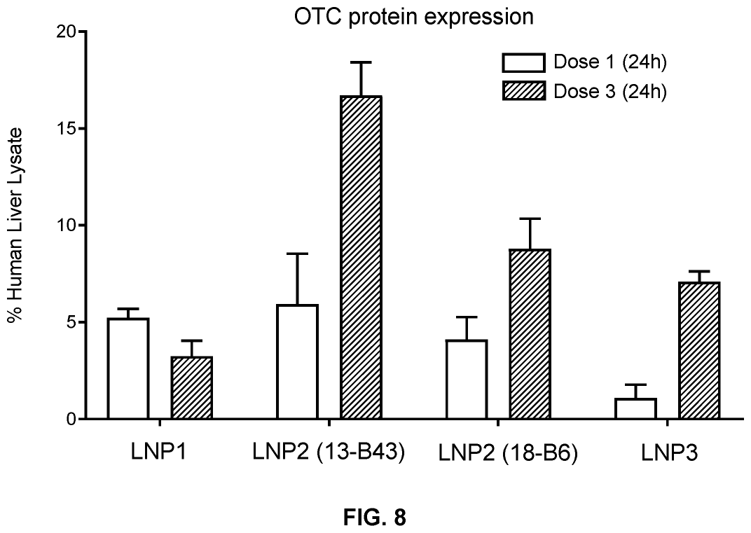

To examine OTC expression levels, blood was collected pre-dose on each dosing day. LNP2 formulations were the most potent, while LNP1 formulations were the least potent (FIG. 8). The highest accumulation of OTC protein was with LNP2 formulations. This data is supported with immunogenicity data which showed no antibodies were produced and there was no accelerated blood clearance. OTC mRNA constructs-LNP2 compositions also had lower repeat-dose MCP-1 levels.

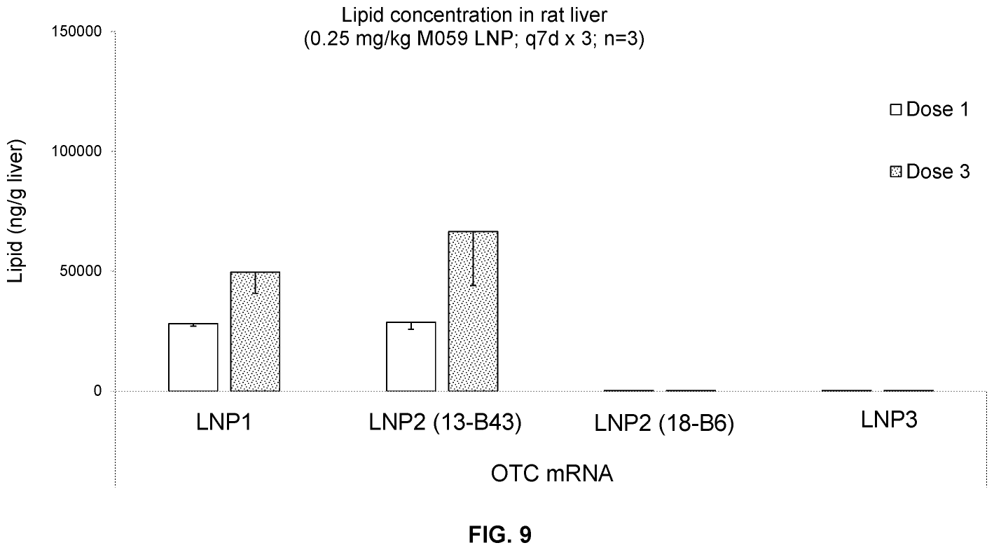

Lipid clearance was quantified 24 h post-dosing by mass spectroscopy. A single dose study showed that LNP1 and LNP2 (13-B43) were present at 14 days post-dose while LNP2 (18-B6) and LNP3 clearly rapidly by 6 h post-dose (data not shown). Repeat dose with OTC mRNA construct-LNP1 or OTC mRNA construct-LNP2 (13-B43) resulted in lipid accumulation in liver (FIG. 9). No accumulation of OTC mRNA construct-LNP2 (18-B6) or OTC mRNA constructs-LNP3 was seen, even upon repeated dose (all levels <LLOQ of 500 ng/g).

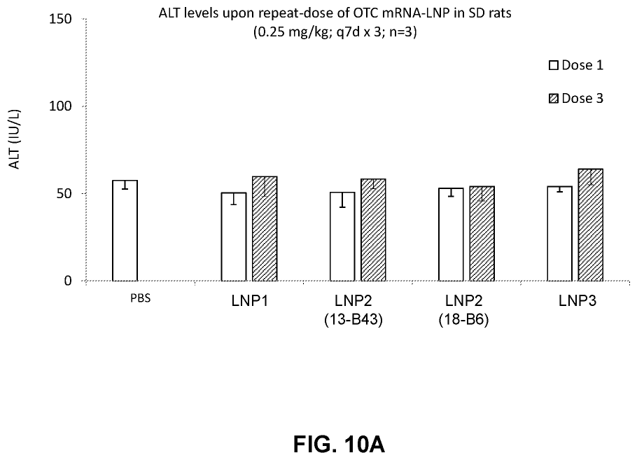

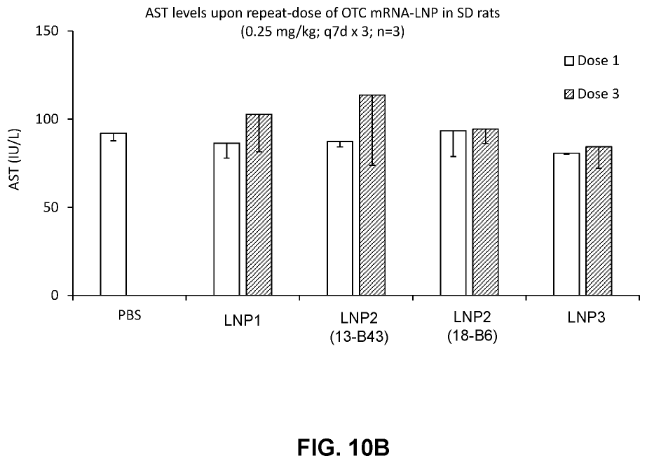

To analyze for markers of liver damage, alanine aminotransferase (ALT) and aspartate aminotransferase (AST) levels were quantified. Serum was collected at 24 h on the first and last day of dosing. There were no significant changes in ALT/AST levels upon repeat dose (0.25 mg/kg administered weekly×3 doses; 0.75 mg/kg total) (FIGS. 10A and 10B). Both LNP1 and LNP2 (13-B43) formulation groups have relatively higher AST compared to the LNP2 (18-B6) and LNP3 formulations after the third dose.

### Example 5. Single Vs. Repeat-Dose Lipid Clearance in Rats

Lipid-clearance following single and repeated-dose administration of OTC mRNA construct-LNP was evaluated. OTC mRNA was formulated in LNP2 (PEG2000-S:13-B43:Cholesterol:DSPC) and administered to rats at 0.25 mg/kg per dose. For single dose, rats were administered the formulation at DO and terminal time points were at 30 min, 1 h, 3 h, 6 h, and 24 h after administration (Table 5A). A high single dose (2 mg/kg) was administered at DO and the terminal time point was D1. For repeated dosing, rats were administered the formulation once every seven days for up to 49 days (day 7, 14, 21, 28, 35, 42, and 49). After the 8th treatment (Day 49) terminal time points were collected at 30 min, 1 h, 3 h, 6 h, and 24 h (Day 50) after dosing. PBS was administered as a control (5 mL/kg) at DO, 7, 14, 21, 28, 35, 42 and 49. The Z-Avg, PDI, and % Encaps of each formulation administered is provided in Table 5B.

TABLE 5A
Single and Repeated Dosing of OTC mRNA construct-LNP2

| | | | | | | Terminal Time |
|---|---|---|---|---|---|---|
| Group | N | Test Article | Dose | | Dose Day | point |
| 1 | 3 | PBS | 5 | mL/kg | D0, 7, 14, 21, 28, 35, 42, 49 | 24 h (D50) |
| 2 | 3 | LNP2 (13-B43) | 2 | mg/kg | D0 (2 mg/kg total) (1.8 mg) | D1 |
| 3 | 3 | | 0.25 | mg/kg | D0 (0.25 mg/kg total) | 30 min |

| | | | |
|---|---|---|---|
| 4 | 3 | | 1 h |
| 5 | 3 | | 3 h |
| 6 | 3 | | 6 h |
| 7 | 3 | | 24 h (D1) |
| 8 | 3 | D0, 7 (0.5 mg/kg total) | D8 |
| | | (0.45 mg) | |
| 9 | 3 | D0, 7, 14 (0.75 mg/kg total) | D15 |
| | | (0.675 mg) | |
| 10 | 3 | D0, 7, 14, 21 (1 mg/kg total) | D22 |
| | | (0.9 mg) | |
| 11 | 3 | D0, 7, 14, 21, 28 | D29 |
| | | (1.25 mg/kg total) (1.125 mg) | |
| 12 | 3 | D0, 7, 14, 21, 28, 35 | D36 |
| | | (1.5 mg/kg total) (1.35 mg) | |
| 13 | 3 | D0, 7, 14, 21, 18, 35, 42 | D43 |
| | | (1.75 mg/kg total) (1.575 mg) | |
| 14 | 3 | D0, 7, 14, 21, 28, 35, 42, 49 | 30 min |
| 15 | 3 | (2 mg/kg total) | 1 h |
| 16 | 3 | | 3 h |
| 17 | 3 | | 6 h |
| 18 | 3 | | 24 hr (D50) |

TABLE 5B
OTC mRNA Construct-LNP2 (13-B43) Formulation Characteristics

| | | | | | | | Input | |
|---|---|---|---|---|---|---|---|---|
| LNP | | | Z-avg | | Encaps | NA | mRNA | Recovery |
| Composition | Payload | | (nm) | PDI | (%) | mg/mL | (mg) | (%) |
| LNP2 (13- | OTC | PF | 80 | 0.11 | 98 | 1.721 | 34.4 | 79 |
| B43) | mRNA | SF | 82 | 0.07 | 95 | | | |
| | construct | F/T | 82 | 0.07 | 96 | | | |

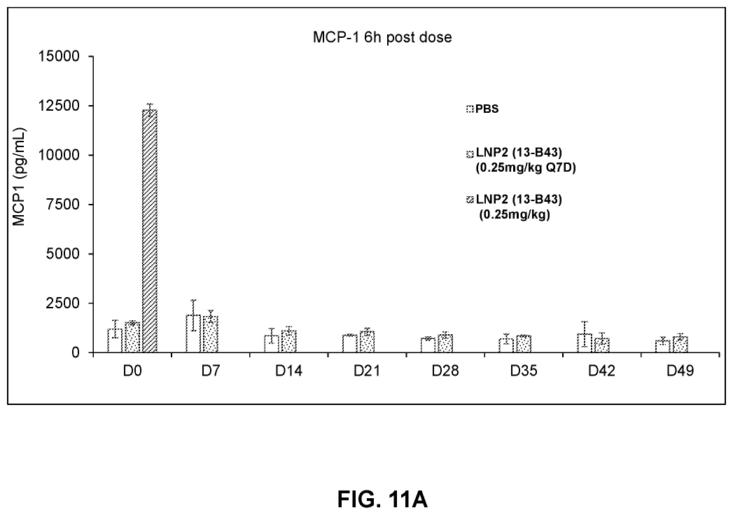

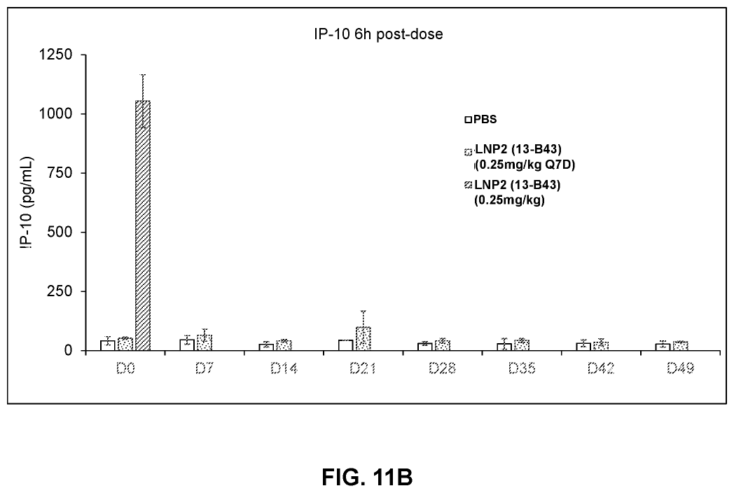

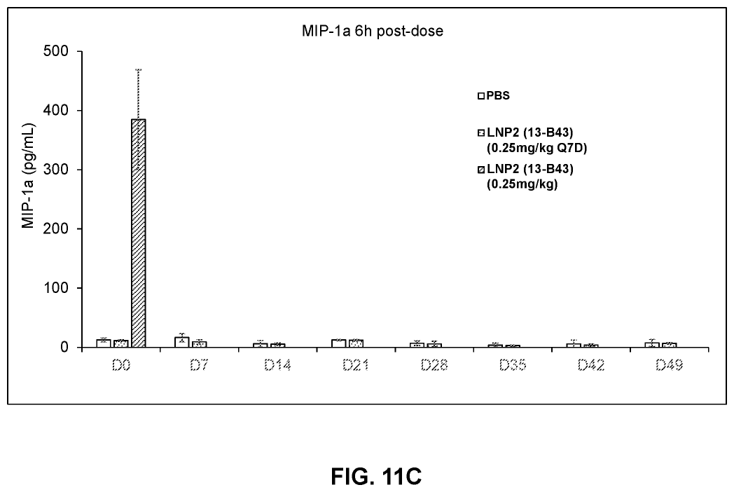

To measure cytokine response, blood was collected at all terminal time points. The cytokines measured were MCP-1, IP-10 and Macrophage inflammatory protein 1α (MIP-1α). There was no cytokine response generated from weekly repeated dose of 0.25 mg/kg (FIGS. 11A-11C). There was a significant cytokine response upon administration of a single dose at 2 mg/kg.

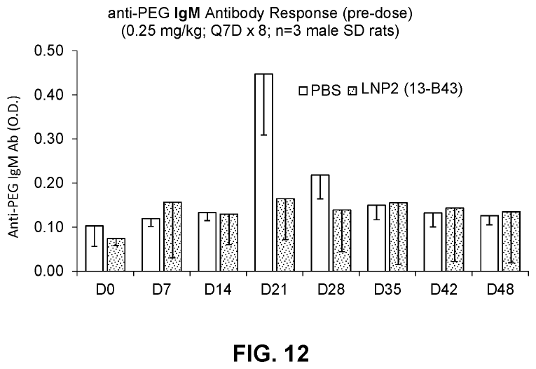

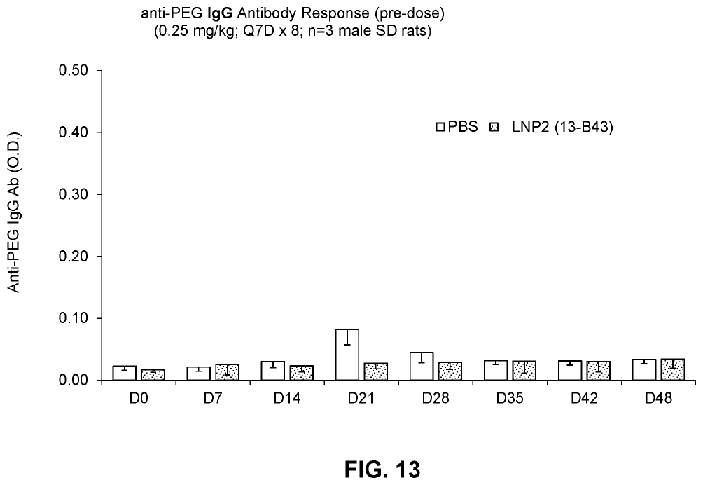

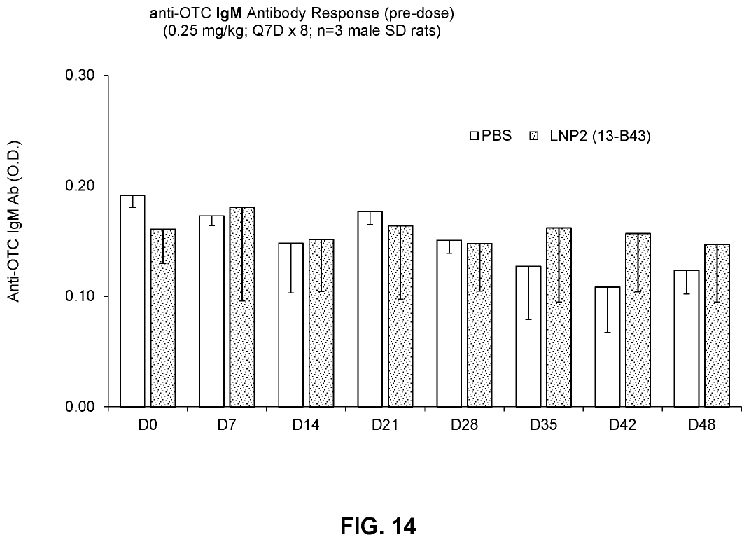

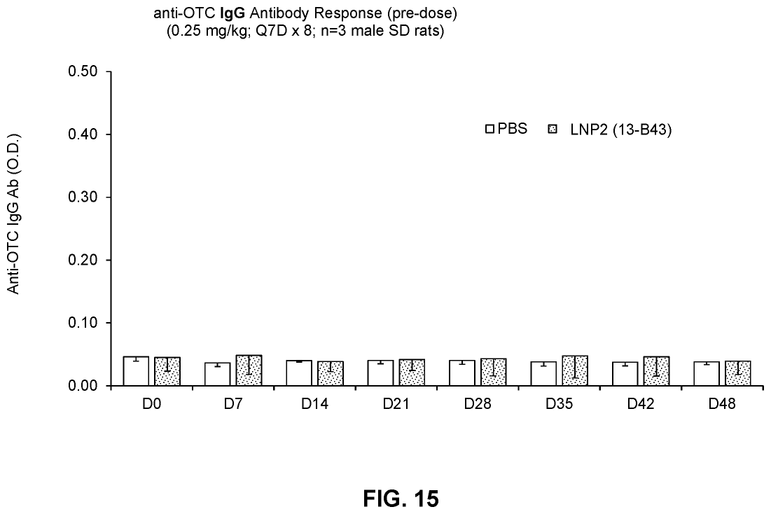

To examine PEG and OTC antibody levels, blood was collected prior to each dose. There was no trend towards increasing levels of anti-PEG IgM with repeated administration (FIG. 12). Similarly, there was no increase in anti-PEG IgG levels with repeated administration of OTC mRNA construct-LNP2 (FIG. 13). No anti-OTC IgM antibodies were detected with repeated administration (FIG. 14). Likewise, no anti-OTC IgG antibodies were detected with repeated administration of OTC mRNA construct-LNP2 (FIG. 15).

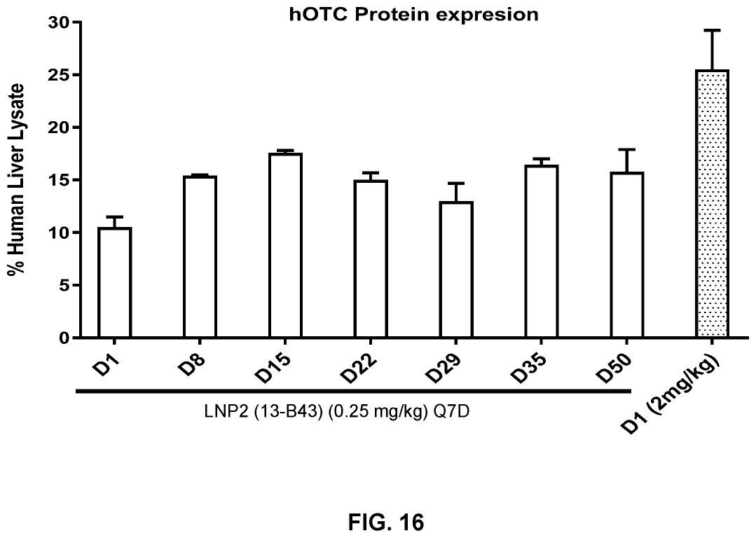

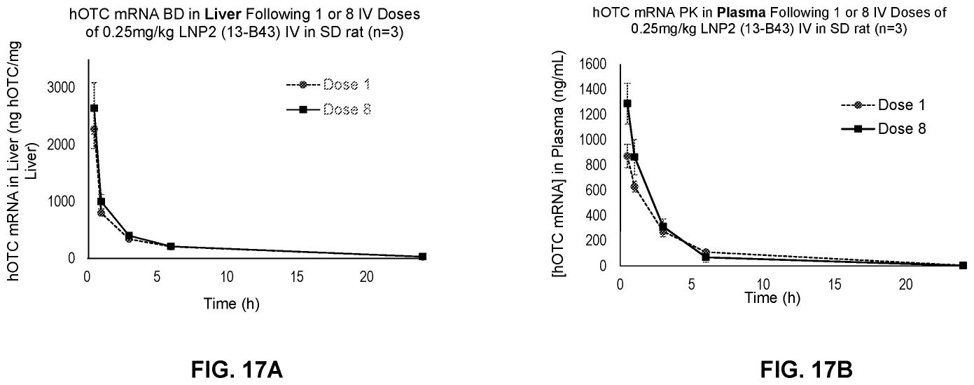

hOTC was also detected in the liver at 24 hours post every dose (FIG. 16). Levels of OTC mRNA in the liver and plasma were quantified over time (30 min, 1 h, 3 h, 6 h, and 24 h) following treatment 1 or 8 (Day 49) (FIGS. 17A and 17B).

### Example 6. Single Dose Range Finding Study in SD Rats

Next the potency and tolerability of LNP1 (PEG2000-C-DMA:13-B43:Cholesterol:DSPC), LNP2 (PEG2000-S:13-B43:Cholesterol:DSPC), and LNP2 (PEG2000-5:18-B6:Cholesterol:DSPC) formulated with OTC mRNA construct were evaluated in a dose response study with SD Rats. Rats were administered OTC mRNA construct-LNP2 at varying concentrations (0.5 mg/kg, 1 mg/kg, or 1.5 mg/kg) and analyzed at for 6 h or 24 h (Table 6A). As a control, some rats were administered 5 mL/kg PBS, 1.5 mg/kg LNP1, or 1.5 mg/kg LNP2. The Z-Avg, PDI, and % Encaps of each formulation administered is provided in Table 6B.

TABLE 6A
Dose Response Study of LNP1 and LNP2

| Group | N | Composition | Dose |
|---|---|---|---|
| 1 | 3 | PBS | 5 mL/kg |
| 2 | | LNP2 (13-B43) | 1.5 mg/kg mRNA equivalence |
| | | | (i.e. ~30 mg/kg lipid) |
| 3 | | LNP2 (18-B6) | 3.0 mg/kg mRNA equivalence |
| | | | (i.e. ~60 mg/kg lipid) |
| 4 | | LNP1 | 1.5 mg/kg |
| 5 | | LNP2 (13-B43) | 0.5 mg/kg |
| 6 | | | 1.0 mg/kg |
| 7 | | | 1.5 mg/kg |
| 8 | | LNP2 (18-B6) | 1.5 mg/kg |
| 9 | | | 3.0 mg/kg |

TABLE 6B
Formulation of LNPs for Dose Range Study

| | | | | | | Endotoxin |
|---|---|---|---|---|---|---|
| | | | | | | (performed |
| | | | | | | at~1 mg/mL |
| | | | | | | mRNA; |
| LNP | | | Z-avg | | Encaps | ~20 mg/mL |
| Composition | Payload | | (nm) | PDI | (%) | lipid) |
| LNP2 (13- | Empty | PF | 62 | 0.13 | N/A | <5 EU/mL |
| B43) | | SF | 79 | 0.25 | | |
| | | F/T | 86 | 0.26 | | |
| LNP2 (18- | Empty | PF | 62 | 013 | N/A | <5 EU/mL |
| B6) | | SF | 79 | 020 | | |
| | | F/T | 83 | 0.18 | | |
| LNP1 | OTC | PF | 79 | 0.27 | 95 | <5 EU/mL |
| | mRNA | SF | 76 | 0.07 | 95 | |
| | construct | F/T | 80 | 0.08 | 98 | |
| LNP2 (13- | OTC | PF | 81 | 0.18 | 95 | <5 EU/mL |
| B43) | mRNA | SF | 83 | 0.07 | 96 | |
| | construct | F/T | 83 | 0.10 | 96 | |
| LNP2 (18- | OTC | PF | 72 | 0.10 | 94 | <5 EU/mL |
| B6) | mRNA | SF | 87 | 0.07 | 93 | |
| | construct | F/T | 86 | 0.09 | 94 | |

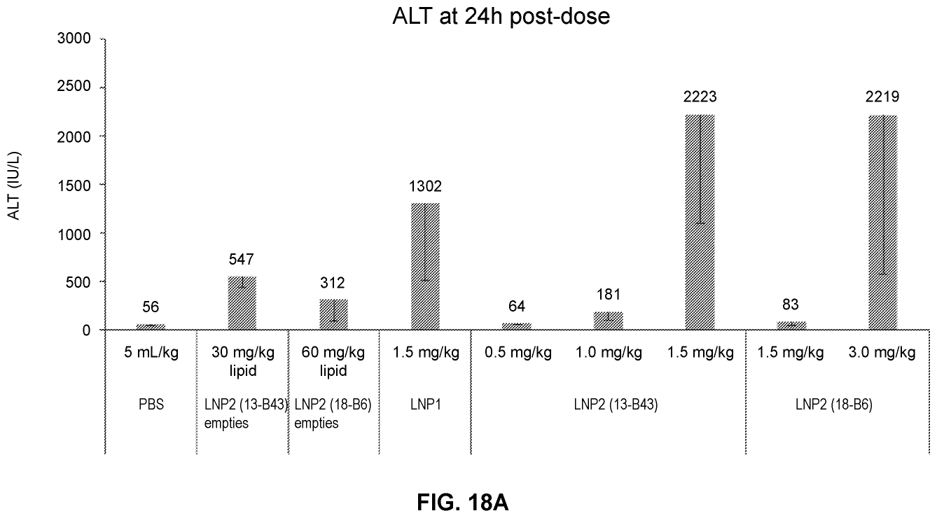

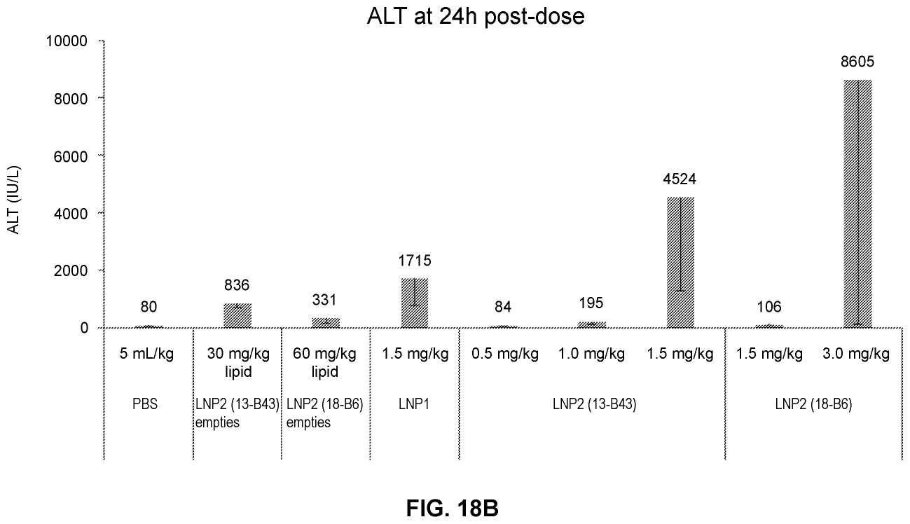

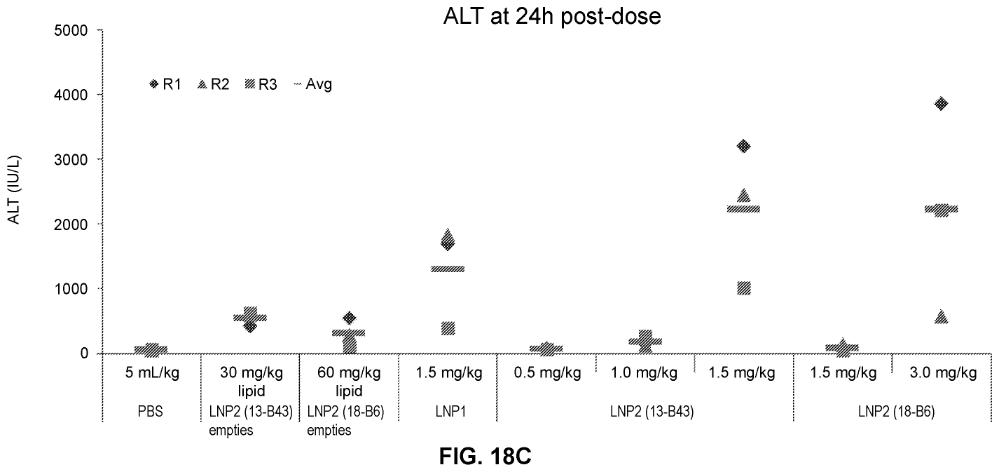

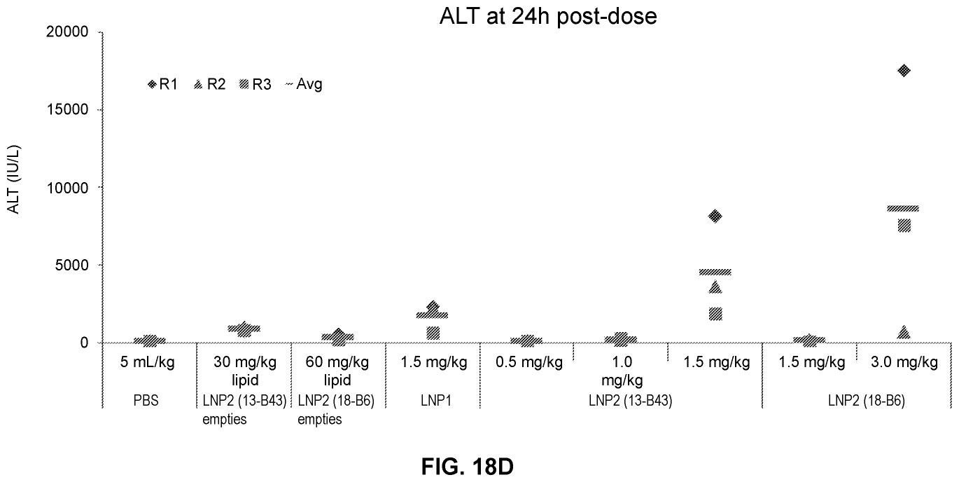

To analyze liver damage, liver samples were collected 24 h post-last dose and ALT, AST, GGT, and total bilirubin levels were analyzed. ALT/AST levels are more elevated compared with mRNA LNPs compared to empties (FIGS. 18A-18D and Table 7). There was a trend of increased ALT/AST levels with increasing dosage of LNP1, LNP2 (13-B43), or LNP2 (18-B6). Administration of 1.5 mg/kg LNP2 (13-B43) induced higher levels of ALT/AST than the same amount of LNP1.

TABLE 7
ALT and AST Levels in Rats Administered with Different Amounts of LNP2

| Animal | Treatment | Dose | | ALT (IU/L) | AST (IU/L) |
|---|---|---|---|---|---|
| 1 | PBS | 5 | mL/kg | 52 | 71 |
| 13 | LNP2 (13-B43) | 0.5 | mg/kg | 72 | 80 |
| 14 | | | | 61 | 95 |
| 15 | | | | 59 | 76 |
| 19 | | 1.5 | mg/kg | 3211 | 8126 |
| 20 | | | | 2450 | 3597 |
| 21 | | | | 1008 | 1848 |
| 22 | LNP2 (18-B6) | 1.5 | mg/kg | 73 | 122 |

| | | |
|---|---|---|
| 23 | 129 | 126 |
| 24 | 46 | 70 |

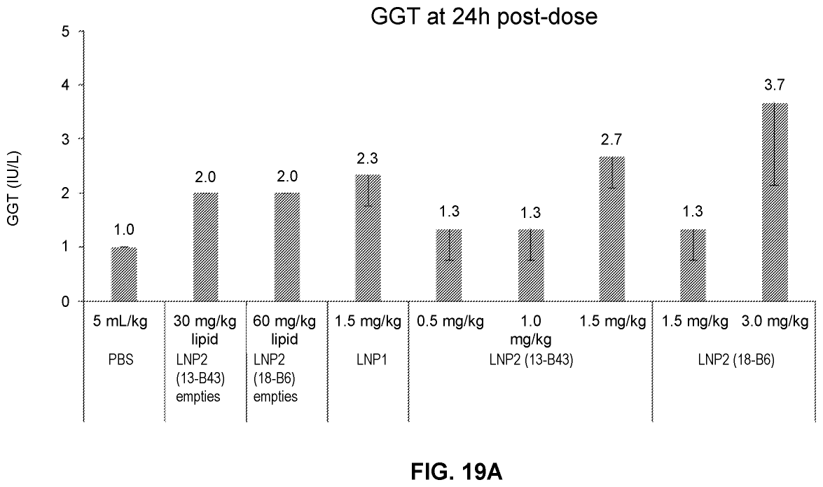

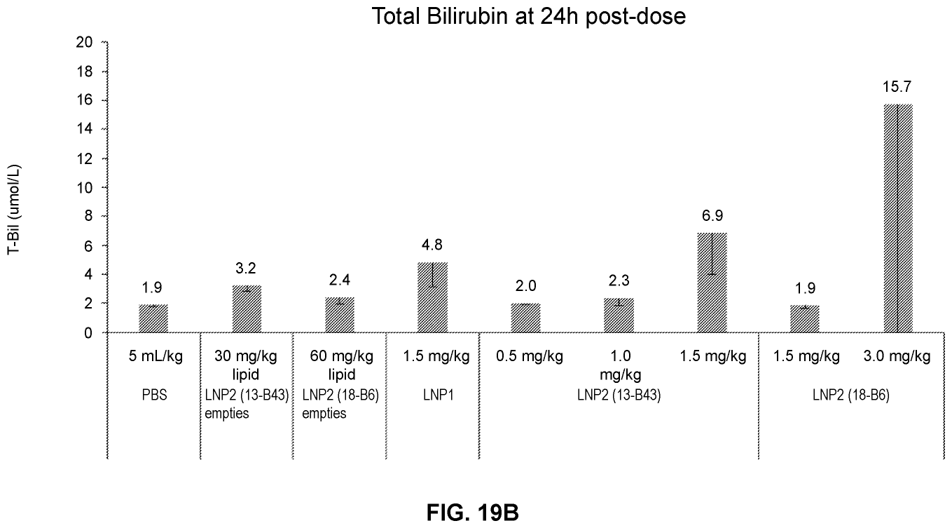

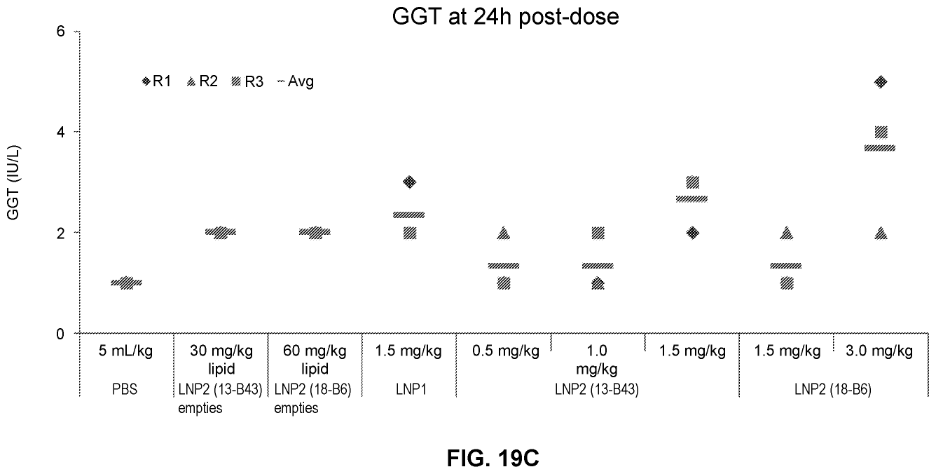

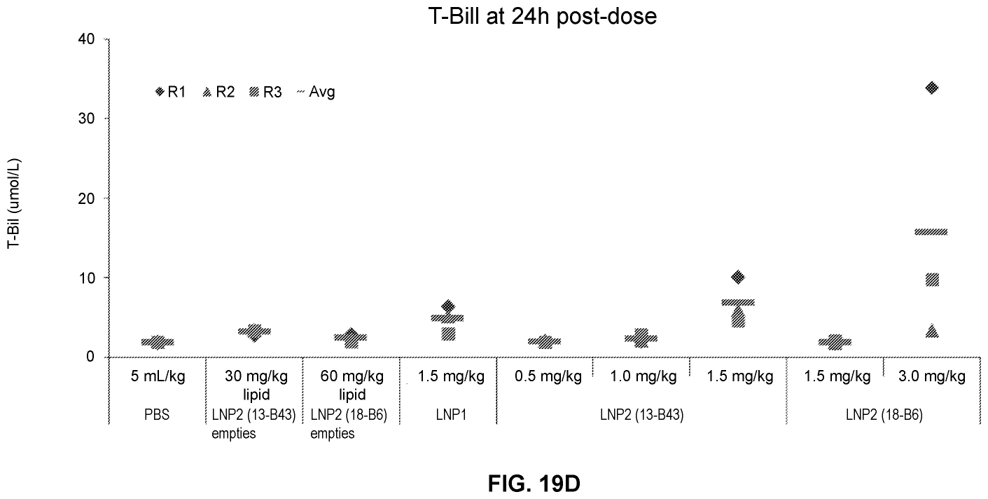

GGT and total bilirubin levels were analyzed in samples taken 24 h post last-dose. There was a trend of increased GGT and total bilirubin levels with increasing dosage of LNP1 or LNP2 OTC mRNA formulations (FIGS. 19A-19D). Administration of 1.5 mg/kg OTC mRNA construct-LNP2 (13-B43) induced similar levels of GGT compared to the same amount of OTC mRNA construct-LNP1. Administration of 1.5 mg/kg OTC mRNA construct-LNP2 induced higher levels of total bilirubin compared to the same amount of OTC mRNA construct-LNP1.

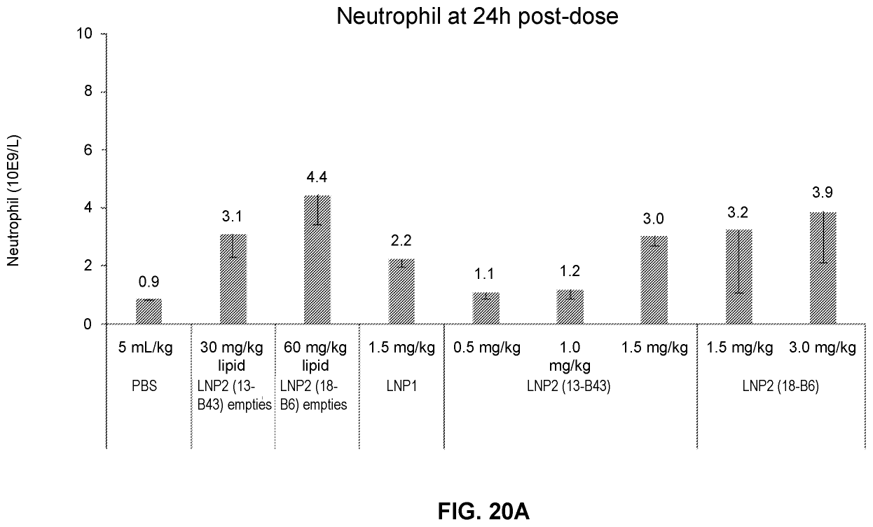

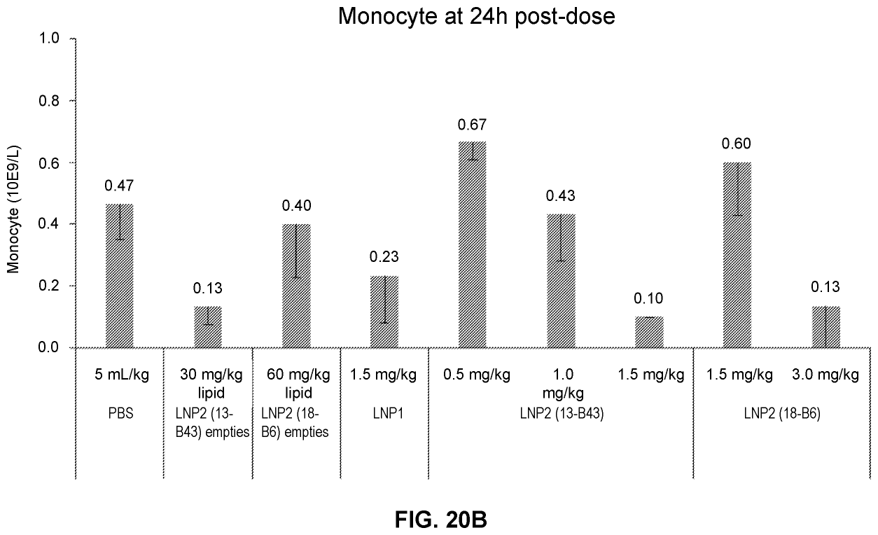

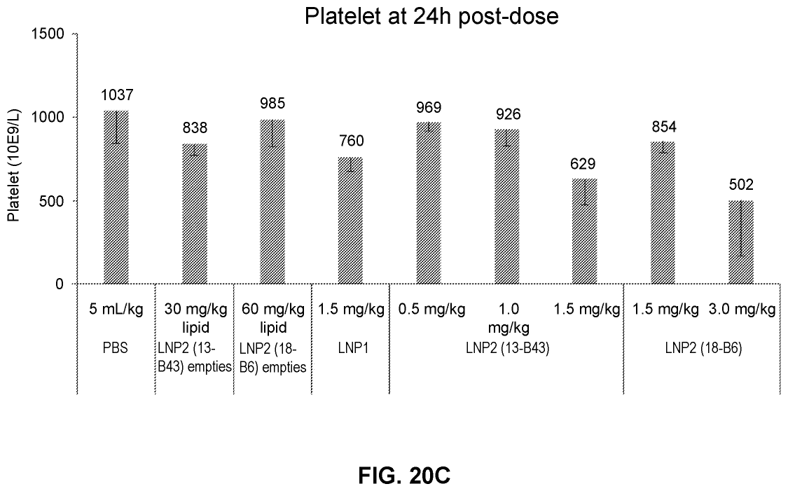

A complete blood count was obtained from blood collect 24 h post last-dose. Rats administered 1.5 mg/kg OTC mRNA construct-LNP1 had similar numbers of neutrophils, monocytes, and platelets compared with rats administered 1.5 mg/kg OTC mRNA construct-LNP2 (13-B43) (FIGS. 20A-20C). Increased dosage of OTC mRNA construct-LNP2 increased the amount of neutrophils but decreased the amount of monocytes and platelets.

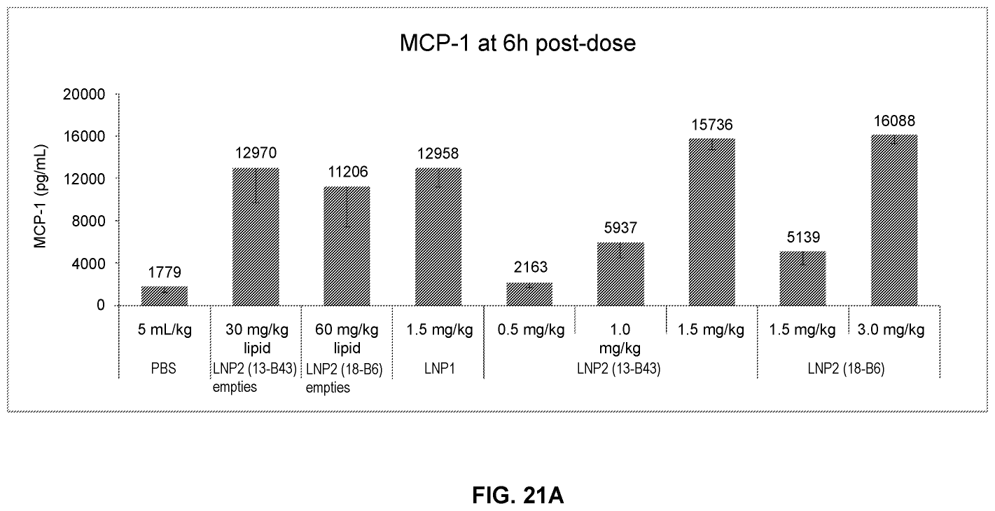

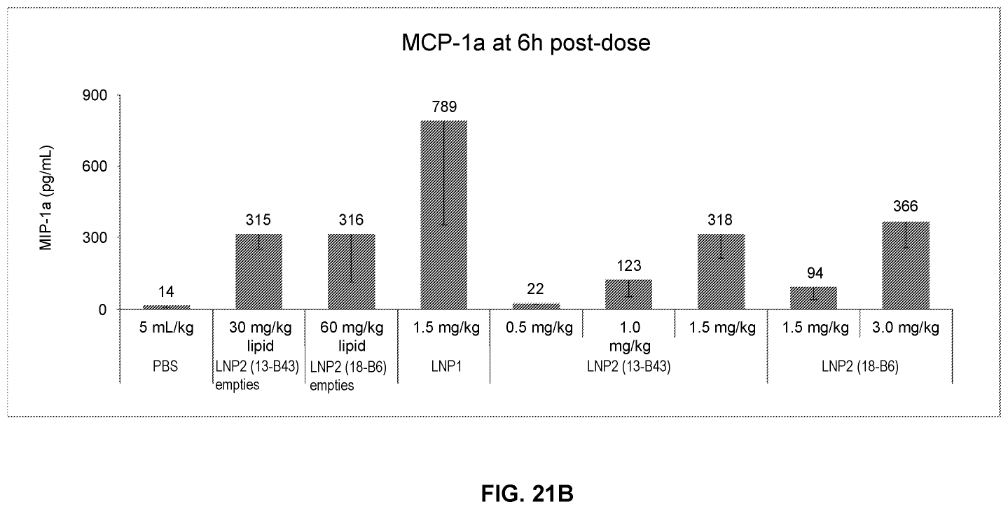

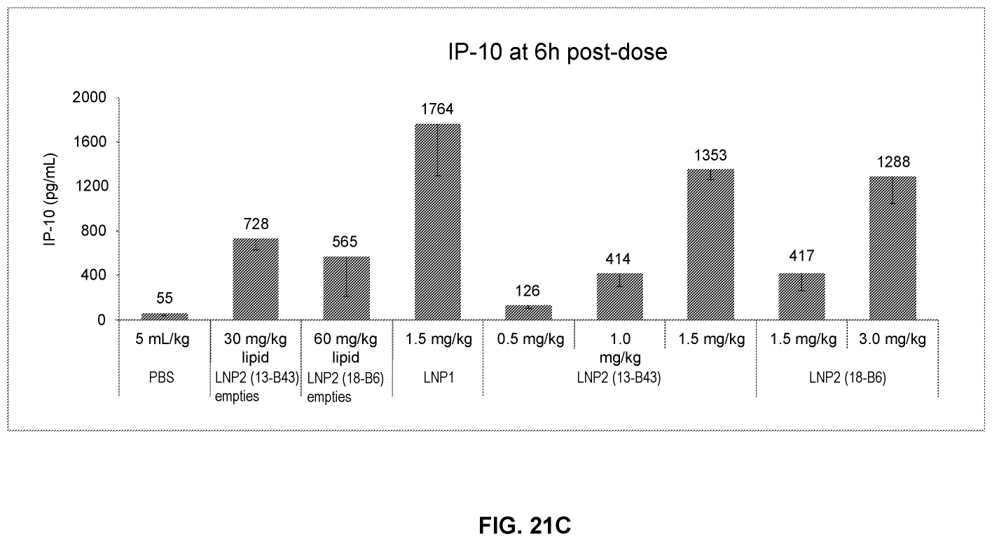

To examine cytokine levels, blood was collected 6 h post-dose and the levels of MCP-1, MIP-1α, and IP-10 were quantified. There was no significant difference in MCP-1 and MIP-1α levels between empties and OTC mRNA construct-LNP compositions (FIGS. 21A-21C). The LNP1 and LNP2 OTC mRNA formulations induced higher levels of IP-10 compared to the empties. There was also a dose-dependent increase in cytokine levels with administration of OTC mRNA construct-LNP2 (13-B43).

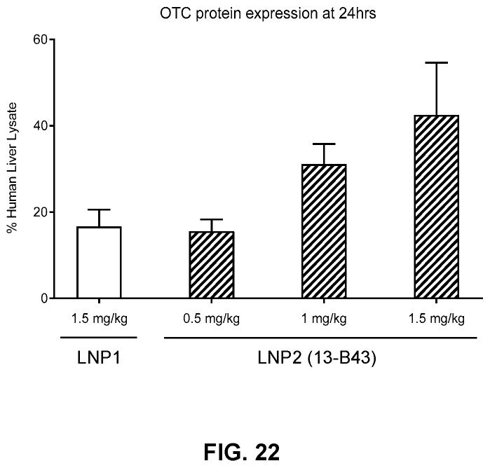

hOTC expression was examined 24 h post last-dose by western blotting. There was an dose-dependent increase in OTC expression with increasing dosage of OTC mRNA construct-LNP2 (13-B43) (FIG. 22). 1.5 mg/kg of OTC mRNA construct-LNP2 (13-B43) provided higher expression of OTC compared to 1.5 mg/kg of OTC mRNA construct-LNP1.

### Example 7. Non-Human Primate Dose Range Study

The potency of LNP1 (PEG2000-C-DMA:13-B43:Cholesterol:DSPC) formulated with OTC mRNA construct was evaluated in a dose response study in non-human primates (NHPs). The OTC mRNA construct included a nucleotide sequence having the 5′, the open reading frame, and the 3′ sequence of SEQ ID NO: 4, a polyA tail length of between 80 nucleotides to 440 nucleotides (i.e., 284 nucleotides), and was pseudouridine (ψ) modified. Non-human primates were administered one dose of OTC mRNA construct-LNP1 at varying concentrations (0.25 mg/kg, 1 mg/kg, 3 mg/kg, or 5 mg/kg) on three different days (day 1, 8, and 15) (Table 8). The results were analyzed at day 16. As a control, the non-human primates were administered 5 mg/kg empty LNP1.

TABLE 8
Formulation of LNPs for Non-Human Primate Dose Range Study

| Group | n | Treatment | Payload | Dose mg/kg |
|---|---|---|---|---|
| 1 | 4 | Control | — | — |
| 2 | | Empty-LNP1 | Empty | 5 |
| | | | | (Equivalent to |
| | | | | Group 6 lipid) |
| 3 | | LNP1 | OTC mRNA | 0.25 |
| 4 | | | construct | 1 |
| 5 | | | | 3 |
| 6 | | | | 5 |

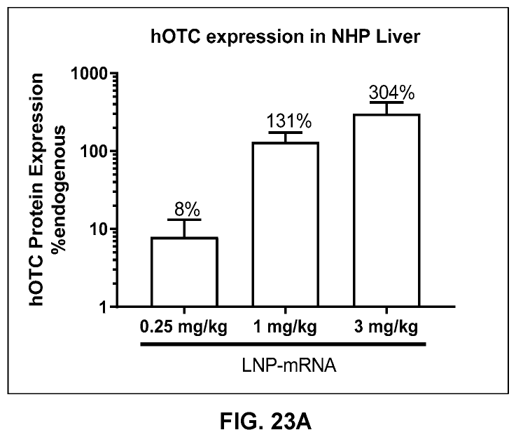

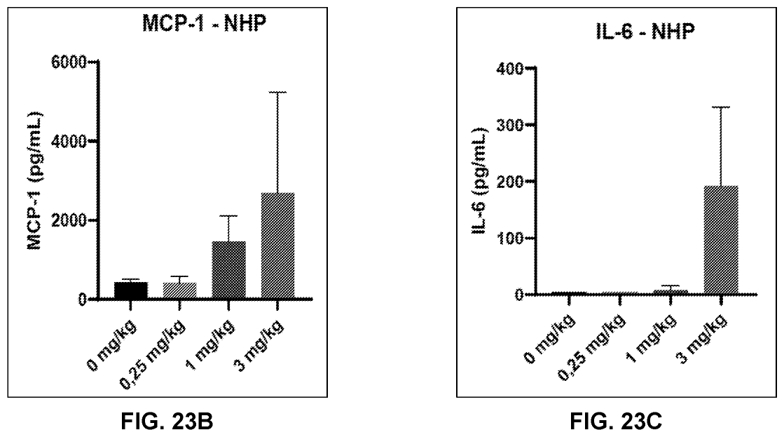

Human OTC expression was analyzed in non-human primate liver samples on day 16. The lowest OTC expression was detected with the 0.25 mg/kg dose and the highest expression was detected with the 3 mg/kg dose, relative to endogenous expression (FIG. 23A). Initial target hOTC expression (8%) was achieved at the lowest dose (0.25 mg/kg).

To examine cytokine levels, samples were collected 6 hrs after the first dose on day 1 and the level of MCP-1 and IL-6 were analyzed (FIGS. 23B & 23C). MCP-1 and IL-6 were not detected in the 0.25 mg/kg dose. Transient elevation of MCP-1 and IL-6 were observed in the 3 mg/kg dose.

These results showed strong hOTC expression with low immune stimulation.

Thus, various aspects are disclosed. The implementations described above and other implementations are within the scope of the following claims. One skilled in the art will appreciate that the present disclosure can be practiced with aspects other than those disclosed. The disclosed aspects are presented for purposes of illustration and not limitation, and the present disclosure is limited only by the claims that follow.

We claim:

1. A polynucleotide construct comprising, from 5′ to 3′:
(a) a 5′ UTR comprising the sequence of SEQ ID NO: 2;
(b) an mRNA sequence comprising an open reading frame (ORF) encoding a functional human ornithine transcarbamylase (OTC), wherein ORF comprises a codon optimized sequence at least about 95% identical to SEQ ID NO: 1; and
(c) a 3′ UTR comprising the sequence of SEQ ID NO: 3.

2. A polynucleotide construct comprising an mRNA sequence comprising an open reading frame (ORF) encoding a functional human ornithine transcarbamylase (OTC), wherein the mRNA sequence comprises a sequence having no more than five nucleic acids different from SEQ ID NO: 4.

3. The polynucleotide construct of claim 2 comprising, from 5′ to 3′:
(a) a 5′ UTR;
(b) the mRNA sequence comprising the ORF encoding the OTC; and
(c) a 3′ UTR.

4. The polynucleotide construct of claim 3, wherein the 5′ UTR comprises the sequence of SEQ ID NO: 2.

5. The polynucleotide construct of claim 3 or 4, wherein the 3′ UTR comprises the sequence of SEQ ID NO: 3.

6. The polynucleotide construct of any one of claims 1-5, wherein the functional OTC comprises the amino acid sequence of SEQ ID NO:7.

7. The polynucleotide construct of any one of claims 1-6, wherein the OFR sequence comprises SEQ ID NO: 1.

8. The polynucleotide construct of any one of claims 1-7, wherein the mRNA sequence has no more than four, three, two, or one nucleic acids different from SEQ ID NO: 4.

9. The polynucleotide construct of any one of claims 1-8 which comprises the sequence of SEQ ID NO: 4.

10. The polynucleotide construct of any one of claims 1-9 which further comprises a 5′ terminal cap.

11. The polynucleotide construct of claim 10, wherein the 5′ terminal cap is a Cap1.

12. The polynucleotide construct of any one of claims 1-11 which further comprises a polyA tail.

13. The polynucleotide construct of claim 12, wherein the polyA tail is between 80 and 1000 nucleic acids long.

14. The polynucleotide construct of claim 12, wherein polyA tail is between 100 and 500 nucleic acids long.

15. The polynucleotide construct of any one of claims 1-14, wherein the mRNA comprises at least one chemically modified uridine.

16. The polynucleotide construct of claim 15, wherein at least about 50%, at least about 60%, at least about 70%, at least about 80%, at least about 85%, at least about 90%, at least about 95%, or about 100% of the uridines are chemically modified.

17. The polynucleotide construct of claim 15 or 16, wherein the chemically modified uridine is selected from the group consisting of pseudouridine (ψ), N1-methyl pseudouridine (N1-me-ψ), and a combination thereof.

18. A composition comprising:
(a) a polynucleotide construct of any one of claims 1-17; and
(b) a delivery agent.

19. The composition of claim 18, wherein the delivery agent comprises a lipid nanoparticle (LNP), a liposome, a polymer, a micelle, a plasmid, a virus, or any combination thereof.

20. The composition of claim 19, wherein the LNP is selected from the group consisting of PEG2000-C-DMA:13-B43:Cholesterol:DSPC, PEG2000-S:13-B43:Cholesterol:DSPC, PEG2000-S:18-B6:Cholesterol:DSPC, and PEG750-C-DLA:18-B6:Cholesterol:DSPC.

21. The composition of claim 19 or 20, wherein the polynucleotide construct is encapsulated in the LNP.

22. The composition of claim 21, wherein the polynucleotide construct is fully encapsulated in the LNP.

23. The composition of claim 22, wherein at least 95% of the polynucleotide construct is encapsulated in the LNP.

24. The composition of any one of claims 18-23 which further comprises a pharmaceutically acceptable carrier.

25. A method for increasing the amount of OTC expression in a cell comprising administering to the cell a composition comprising the polynucleotide construct of any one of claims 1-17 or the composition of any one of claims 18-24.

26. The method of claim 25, wherein the cell is a liver cell.

27. A method for treating or reducing the symptoms associated with ornithine transcarbamylase deficiency (OTCD) comprising administering to a subject in need thereof a therapeutically effective amount of a composition comprising the polynucleotide construct of any one of claims 1-17 or the composition of any one of claims 18-24.

28. A method for treating or reducing the risk of hyperammonemia in a subject with OTCD comprising administering to a subject in need thereof a therapeutically effective amount of a composition comprising the polynucleotide construct of any one of claims 1-17 or the composition of any one of claims 18-24.

29. An expression cassette comprising a sequence having at least 85%, at least 90%, at least 95%, at least 96%, at least 97%, at least 98%, at least 99%, or 100% sequence identity to SEQ ID NO: 8.

30. The expression cassette of claim 29 which further comprises a promoter.

31. The expression cassette of claim 30, wherein the promoter is a T7 promoter.

32. A plasmid comprising the expression cassette of any one of claims 29-31.

33. A host cell comprising the expression cassette of any one of claims 29-31, or the plasmid of claim 32.

34. Use of the polynucleotide construct of any one of claims 1-17, or the composition of any one of claims 18-24, or the expression cassette of claim any one of claims 29-31, or the plasmid of claim 32, or the host cell of claim 33, for the manufacture of a medicament for the treatment of OTCD in a subject in need thereof.

35. Use of the polynucleotide construct of any one of claims 1-17, or the composition of any one of claims 18-24, or the expression cassette of claim any one of claims 29-31, or the plasmid of claim 32, or the host cell of claim 33, for the manufacture of a medicament for the treatment of or for reducing the risk of hyperammonemia in a subject with OTCD.

36. A method for the in vivo delivery of a nucleic acid, the method comprising:
administering to a mammalian subject the polynucleotide construct of any one of claims 1-17, or the composition of any one of claims 18-24, or the expression cassette of any one of claims 29-31, or the plasmid of claim 32, or the host cell of claim 33.

37. A method for treating a disease or disorder in a mammalian subject in need thereof, the method comprising: administering to the mammalian subject a therapeutically effective amount of the polynucleotide construct of any one of claims 1-17, or the composition of any one of claims 18-24, or the expression cassette of any one of claims 29-31, or the plasmid of claim 32, or the host cell of claim 33.

38. The method of claim 37, wherein the disease or disorder is a urea cycle disorder.
